(19) United States
(12) Patent Application Publication
(10) Pub. No.: US 2023/0017948 A1
(43) Pub. Date: Jan. 19, 2023

(54) COMBINATION THERAPY USING FABP5 INHIBITORS WITH TAXANES FOR TREATMENT OF CANCER

(71) Applicants: Iwao OJIMA, Port Jefferson, NY (US); Martin Martin KACZOCHA, Dix Hills, NY (US); Gregory CARBONETTI, Bloomfield, NJ (US); The Research Foundation for The State University of New York, Albany, NY (US)

(72) Inventors: Iwao Ojima, Port Jefferson, NY (US); Martin Kaczocha, Dix Hills, NY (US); Gregory Carbonetti, Bloomfield, NJ (US)

(73) Assignee: The Research Foundation for The State University of New York, Albany, NY (US)

(21) Appl. No.: 17/779,214

(22) PCT Filed: Nov. 24, 2020
(86) PCT No.: PCT/US20/61918

§ 371 Date: May 24, 2022

Related U.S. Application Data
(60) Provisional application No. 62/940,006, filed on Nov. 25, 2019.

(51) Int. Cl. A61K 31/216; A61K 31/337; A61K 45/06; A61P 35/00; C07C 69/757

(52) U.S. Cl. CPC A61K 31/216; A61K 31/337; A61K 45/06; A61P 35/00; C07C 69/757; C07C 2601/04; C07C 2603/18

(57) ABSTRACT

This invention provides a method of treating a subject afflicted with a cancer comprising periodically administering to the subject an amount of a FABP5 inhibitor and an amount of an anticancer therapy, wherein the amounts when taken together are effective to treat the subject. This invention also provides FABP5 inhibitors for use as an add-on therapy or in combination with an anticancer therapy or in treating a subject afflicted with a cancer. This invention also provides the use of a FABP5 inhibitor in the manufacturing of a medicament for use in combination or as an add on with an anticancer therapy in treating a subject afflicted with cancer, wherein the FABP5 inhibitor and the anticancer therapy are administered simultaneously, contemporaneously or concomitantly. This invention also provides a pharmaceutical composition comprising an amount of a FABP5 inhibitor and an amount of an anticancer therapy for use in treating a subject afflicted with a cancer, wherein the FABP5 inhibitor and an anticancer therapy are administered sequentially or simultaneously.

This application claims priority of U.S. Provisional Application No. 62/940,006, filed Nov. 25, 2019, the contents of which are hereby incorporated by reference.

Throughout this application, certain publications are referenced in parentheses. Full citations for these publications may be found immediately preceding the claims. The disclosures of these publications in their entireties are hereby incorporated by reference into this application in order to describe more fully the state of the art to which this invention relates.

### BACKGROUND OF THE INVENTION

Despite advances in anti-androgen and chemotherapeutic interventions, prostate cancer (PCa) remains the second leading cause of cancer-related death in men in the United States (Bray, F. et al. 2018). After anti-androgen therapy, metastatic PCa often becomes castration-resistant and incurable, highlighting the need to develop next-generation therapeutics to treat aggressive metastatic PCa (Frieling, J. S. et al. 2015). Adenocarcinomas of the prostate utilize lipids to fuel their growth, and dysregulated lipid metabolism is observed in PCa (Deep, G. & Schlaepfer, I. 2016; Zadra, G. et al. 2013). The nuclear receptor peroxisome proliferator-activated receptor γ (PPARγ), which regulates the expression of proangiogenic genes, is overexpressed in metastatic prostate adenocarcinomas and is associated with reduced patient survival (Forootan, F. S. et al. 2014; Ahmad, I. et al. 2016; Bao, Z. et al. 2013).

Fatty acid-binding protein 5 (FABP5) is a member of a class of intracellular lipid chaperones that transports fatty acids to PPARγ, leading to increased expression of proangiogenic factors including vascular endothelial growth factor, which can result in a metastatic phenotype (Forootan, F. S. et al. 2014; Morgan, E. A. et al. 2008; Forootan, F. S. et al. 2016; Adamson, J. et al. 2003; Furuhashi, M. & Hotamisligil, G. S. 2008; Jing, C. et al. 2000). The normal prostate lacks FABP5 expression but it becomes highly upregulated in PCa and the degree of its upregulation correlates with increasing Gleason scores, indicating that advanced metastatic prostate tumors express the highest levels of FABP5 (Forootan, F. S. et al. 2014; Morgan, E. A. et al. 2008; Jing, C. et al. 2000; Fujita, K. et al. 2017). Mirroring this expression pattern, PCa cell-lines with low metastatic potential lack FABP5 expression, whereas PCa cell-lines with high metastatic potential demonstrate elevated FABP5 expression levels (Forootan, F. S. et al. 2014; Kawaguchi, K. et al. 2016). Introduction of FABP5 to PCa cell-lines with low metastatic potential enhances cell migration, invasion, and tumor formation, while its inhibition in PCa cell lines with high metastatic potential attenuates these features (Bao, Z. et al. 2013; Forootan, F. S. et al. 2016; Kawaguchi, K. et al. 2016). This positions FABP5 as a potential therapeutic target to treat PCa.

Herein, we show that FABP5 inhibitors potentiate the cytotoxic and tumor-suppressive effects of antitumor therapies.

Combination Therapy

The administration of two drugs to treat a given condition, such as cancer, raises a number of potential problems. In vivo interactions between two drugs are complex. The effects of any single drug are related to its absorption, distribution, and elimination. When two drugs are introduced into the body, each drug can affect the absorption, distribution, and elimination of the other and hence, alter the effects of the other. For instance, one drug may inhibit, activate or induce the production of enzymes involved in a metabolic route of elimination of the other drug (Guidance for Industry, 1999). In one example, combined administration of fingolimod and interferon (IFN) has been experimentally shown to abrogate the clinical effectiveness of either therapy (Brod, 2000). In another experiment, it was reported that the addition of prednisone in combination therapy with IFN-β antagonized its up-regulator effect. Thus, when two drugs are administered to treat the same condition, it is unpredictable whether each will complement, have no effect on, or interfere with, the therapeutic activity of the other in a human subject.

Not only may the interaction between two drugs affect the intended therapeutic activity of each drug, but the interaction may increase the levels of toxic metabolites (Guidance for Industry, 1999). The interaction may also heighten or lessen the side effects of each drug. Hence, upon administration of two drugs to treat a disease, it is unpredictable what change will occur in the negative side profile of each drug. In one example, the combination of natalizumab and interferon β-1a was observed to increase the risk of unanticipated side effects. (Vollmer, 2008; Rudick, 2006; Kleinschmidt-DeMasters, 2005; Langer-Gould, 2005)

Additionally, it is difficult to accurately predict when the effects of the interaction between the two drugs will become manifest. For example, metabolic interactions between drugs may become apparent upon the initial administration of the second drug, after the two have reached a steady-state concentration or upon discontinuation of one of the drugs (Guidance for Industry, 1999).

Therefore, the state of the art at the time of filing is that the effects of combination therapy of two drugs, in particular a FABP5 inhibitor and a taxane, such as docetaxel or cabazitaxel, cannot be predicted until the results of a combination study are available.

### SUMMARY OF THE INVENTION

This invention provides a method of treating a subject afflicted with cancer comprising periodically administering to the subject an amount of a FABP5 inhibitor and an amount of an anticancer therapy, wherein the amounts when taken together are effective to treat the subject.

This invention also provides FABP5 inhibitors for use as an add-on therapy or in combination with an anticancer therapy or in treating a subject afflicted with a cancer.

This invention also provides the use of a FABP5 inhibitor in the manufacturing of a medicament for use in combination or as an add on with an anticancer therapy in treating a subject afflicted with cancer, wherein the FABP5 inhibitor and the anticancer therapy are administered simultaneously, contemporaneously or concomitantly.

This invention also provides a pharmaceutical composition comprising an amount of a FABP5 inhibitor and an amount of an anticancer therapy for use in treating a subject afflicted with a cancer, wherein the FABP5 inhibitor and an anticancer therapy are administered sequentially or simultaneously.

### BRIEF DESCRIPTION OF THE FIGURES

FIG. 1: SBFI-102/SBFI-103 structures and in vitro affinities (Ki, μM), and docetaxel/cabazitaxel structures. The chemical structures and Ki values of (A) SBFI-102 and (B) SBFI-103 are shown (Yan et al. 2018). The chemical structures for (C) docetaxel and (D) cabazitaxel.

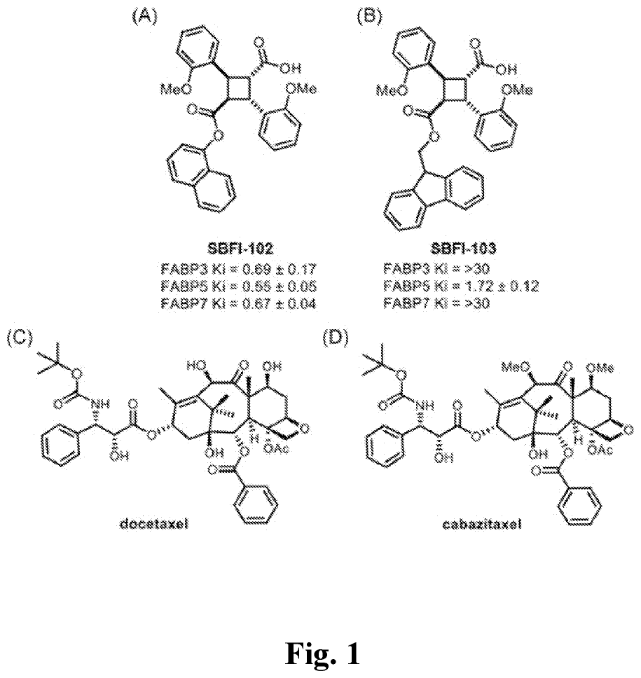

FIGS. 2A-2B: Cytotoxicity of SBFI-102 (FIG. 2A) and SBFI-103 (FIG. 2B) in PC3, DU-145, 22Rv1, RWPE-1, and WI-38 cells (n 3). SBFI-102 (FIG. 2A) produced cytotoxicity in PC3, DU-145, 22Rv1, RWPE-1, and WI-38 cells with IC50 values of 11.4, 8.9, 10.1, 26.0, and 29.4 μM, respectively (n 3). SBFI-103 (FIG. 2B) produced cytotoxicity in PC3, DU-145, 22Rv1, RWPE-1, and WI-38 cells with IC50 values of 6.3, 3.3, 3.1, 20.6, and 29.6 μM, respectively (n 3).

FIGS. 3A-3C: Cytotoxicity of docetaxel in PC3, DU-145, and 22Rv1 cells. Docetaxel produced cytotoxicity in (A) PC3, (B) DU-145, and (C) 22Rv1 cells with IC50 values of 1.9, 0.8, and 0.3 nM, respectively (n 3). IC50, half-maximal inhibitory concentration.

FIGS. 4A-4C: Cytotoxicity of cabazitaxel in PC3, DU-145, and 22Rv1 cells. Cabazitaxel produced cytotoxicity in (A) PC3, (B) DU-145, and (C) 22Rv1 cells with IC50 values of 1.6, 0.2, and 0.3 nM, respectively (n 3). IC50, half-maximal inhibitory concentration.

FIGS. 5A-5F: Cytotoxicity of PC3, DU-145, and 22Rv1 cells following combinatorial treatment with docetaxel and SBFI-102 or SBFI-103. Cytotoxicity of PC3 cells incubated with docetaxel in the presence of A) SBFI-102 or B) SBFI-103 (n 3). Cytotoxicity of DU-145 cells incubated with docetaxel in the presence of C) SBFI-102 or D) SBFI-103 (n 3). Cytotoxicity of 22Rv1 cells incubated with docetaxel in the presence of E) SBFI-102 or F) SBFI-103 (n 3).

FIG. 6A-6F: Cytotoxicity of PC3, DU-145, and 22Rv1 cells following combinatorial treatment with cabazitaxel and SBFI-102 or SBFI-103. Cytotoxicity of PC3 cells incubated with cabazitaxel in the presence of A) SBFI-102 or B) SBFI-103 (n 3). Cytotoxicity of DU-145 cells incubated with cabazitaxel in the presence of C) SBFI-102 or D) SBFI-103 (n 3). Cytotoxicity of 22Rv1 cells incubated with cabazitaxel in the presence of E) SBFI-102 or F) SBFI-103 (n 3).

FIG. 7A-7D: Inhibition of subcutaneous tumor growth by docetaxel or FABP5 inhibitors. PC3 cells (1×10⁶) were implanted subcutaneously into male BALB/c nude mice. From day 15 onwards, mice were treated with vehicle, SBFI-102 (20 mg/kg, daily), SBFI-103 (20 mg/kg, daily), or docetaxel (5 mg/kg or 10 mg/kg, weekly). A) Tumor growth over the time course of treatments. B-D) Tumor volumes at days 25, 30, and 35, respectively. *P<0.05 versus vehicle treatment; **P<0.01 versus vehicle treatment; ***P<0.001 versus vehicle treatment; #P<0.05 versus 10 mg/kg docetaxel treatment; ##P<0.01 versus 10 mg/kg docetaxel treatment; (n=5).

FIG. 8A-8D: Inhibition of subcutaneous tumor growth by docetaxel and FABP5 inhibitors. PC3 cells (1×10⁶) were implanted subcutaneously into male BALB/c nude mice. From day 15 onwards, mice were treated with vehicle, SBFI-102 (20 mg/kg, daily) in combination with docetaxel (5 mg/kg, weekly), SBFI-103 (20 mg/kg, daily) in combination with docetaxel (5 mg/kg, weekly), or docetaxel (5 mg/kg or 10 mg/kg, weekly). A) Tumor growth over the time course of treatments. B-D) Tumor volumes at days 25, 30, and 35, respectively. **P<0.01 versus vehicle treatment; ***P<0.001 versus vehicle treatment; #P<0.05 versus 10 mg/kg docetaxel treatment; NS versus 10 mg/kg docetaxel treatment; (n=5).

### DETAILED DESCRIPTION OF THE INVENTION

This invention provides a method of treating cancer in a subject comprising administering to the subject an effective amount of a FABP5 inhibitor with an anticancer therapy.

This invention also provides a method comprising periodically administering to the subject an amount of a FABP5 inhibitor and an anticancer therapy, wherein the amounts when taken together are effective to treat the subject.

In an embodiment, the amount of FABP5 inhibitor, and the amount of an anticancer therapy when administered together is more effective to treat the subject than when each agent at the same amount is administered alone.

In an embodiment, the subject is receiving the anticancer therapy prior to initiating FABP5 inhibitor therapy.

In an embodiment, the subject is receiving FABP5 inhibitor therapy prior to initiating an anticancer therapy.

In an embodiment, the FABP5 inhibitor, and the anticancer therapy are administered sequentially.

In an embodiment, the FABP5 inhibitor is administered first, followed by administration of the anticancer therapy.

In an embodiment, an anticancer therapy is administered first, followed by administration of the FABP5 inhibitor.

In an embodiment, the FABP5 inhibitor, and the anticancer therapy are administered simultaneously.

In an embodiment, the FABP5 inhibitor is administered orally. In an embodiment, the FABP5 inhibitor is administered intravenously. In an embodiment, the FABP5 inhibitor is administered intraperitoneally.

In an embodiment, the anticancer therapy is a taxane.

In an embodiment, the taxane is administered intravenously. In an embodiment, the taxane is administered intraperitoneally.

In an embodiment, the cancer expresses FABP5.

In an embodiment, the cancer overexpresses FABP5.

In an embodiment, the cancer is prostate cancer. In an embodiment, the cancer is skin cancer. In an embodiment, the cancer is breast cancer. In an embodiment, the cancer is hepatocellular carcinoma. In an embodiment, the cancer is cervical cancer.

In a preferred embodiment, the cancer is prostate cancer. In another preferred embodiment, the cancer is drug-resistant prostate cancer. In another preferred embodiment, the cancer is metastatic prostate cancer.

In an embodiment, the FABP5 inhibitor has the structure:

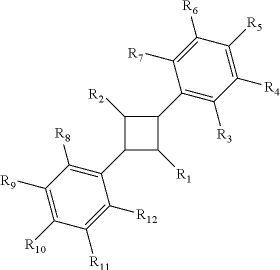

wherein

R₁ and R₂ are different and are each —C(═O)R₁₃, —C(═O)OR₁₃, —C(═O)O-alkyl-R₁₃, —C(═O)NR₁₃R₁₄, -alkyl-C(═O)R₁₃, -alkyl-C(═O)OR₁₃, -alkyl-C(═O)NR₁₃R₁₄, -alkyl-OC(═O)OR₁₃, -alkyl-OC(═O)R₁₃, -alkyl-NR₁₃R₁₄, -alkyl-NHC(═O)R₁₃, -alkyl-NHC(═O)OR₁₃, -alkyl-NHC(═O)R₁₃, -alkyl-NHC(═O)NR₁₃R₁₄, -alkyl-NHC(═S)NR₁₃R₁₄, -alkyl-NHC(═NR₁₃)NR₁₃R₁₄, —C(—OH) C(═O)OR₁₃, C(═O)C(═O)OR₁₃ or —C═C—R₁₃,
wherein R₁₃ and R₁₄ are each, independently, H, CF₃, C₁₋₁₀ alkyl, C₂₋₁₀ alkenyl, C₂₋₁₀ alkynyl, heteroalkyl, cycloalkyl, cycloheteroalkyl, aryl, heteroaryl, heterocyclyl or combine to form a cycloalkyl or heterocyclyl;
   R₃, R₄, R₅, R₆, R₇, R₈, R₉, R₁₃, R₁₁ and R₁₂ are each independently, H, halogen, —NO₂, —CN, —NHR₁₅, —NR₁₅R₁₆, —SR₁₅, —SO₂R₁₅, —OR₁₅, —CO₂R₁₅, CF₃, -alkyl-NR₁₅R₁₆, -alkyl-OR₁₅, C₁₋₁₉ alkyl, C₂₋₁₀ alkenyl, C₂₋₁₀ alkynyl, aryl, heteroaryl, or heterocyclyl;
   wherein R₁₅ and R₁₆ are each, independently, H, CF₃, C₁₋₁₀ alkyl, C₂₋₁₀ alkenyl, C₂₋₁₀ alkynyl, heteroalkyl, cycloheteroalkyl, aryl, heteroaryl, or heterocyclyl;
   or an enantiomer or racemate thereof;
   or a pharmaceutically acceptable salt thereof.

In an embodiment, the compound has the stereochemistry of structure I

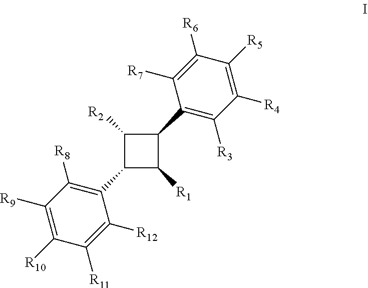

then
R₁ and R₂ are different and are each —C(═O)R₁₃, —C(═O)OR₁₃, —C(═O)O-alkyl-R₁₃, —C(═O)NR₁₃R₁₄, -alkyl-C(═O)R₁₃, -alkyl-C(═O)OR₁₃, -alkyl-C(═O)NR₁₃R₁₄, -alkyl-OC(═O)OR₁₃, -alkyl-OC(═O)R₁₃, -alkyl-NR₁₃R₁₄, -alkyl-NHC(═O)R₁₃, -alkyl-NHC(═O)OR₁₃, -alkyl-NHC(═O)R₁₃, -alkyl-NHC(═O)NR₁₃R₁₄, -alkyl-NHC(═S)NR₁₃R₁₄, -alkyl-NHC(═NR₁₃)NR₁₃R₁₄, —C(—OH) C(═O)OR₁₃, C(═O)C(═O)OR₁₃ or —C═C—R₁₃,
wherein R₁₃ and R₁₄ are each, independently, H, CF₃, C₁₋₁₀ alkyl, C₂₋₁₀ alkenyl, C₂₋₁₀ alkynyl, heteroalkyl, cycloalkyl, cycloheteroalkyl, aryl, heteroaryl, heterocyclyl or combine to form a cycloalkyl or heterocyclyl;
   R₃, R₄, R₅, R₆, R₇, R₈, R₉, R₁₃, R₁₁ and R₁₂ are each independently, H, halogen, —NO₂, —CN, —NHR₁₅, —NR₁₅R₁₆, —SR₁₅, —SO₂R₁₅, —OR₁₅, —CO₂R₁₅, CF₃, -alkyl-NR₁₅R₁₆, -alkyl-OR₁₅, C₁₋₁₀ alkyl, C₂₋₁₀ alkenyl, C₂₋₁₀ alkynyl, aryl, heteroaryl, or heterocyclyl;
   wherein R₁₅ and R₁₆ are each, independently, H, CF₃, C₁₋₁₉ alkyl, C₂₋₁₃ alkenyl, C₂₋₁₀ alkynyl, heteroalkyl, cycloheteroalkyl, aryl, heteroaryl, or heterocyclyl;
   or an enantiomer or racemate thereof;
   or a pharmaceutically acceptable salt thereof.

In an embodiment, the compound has the stereochemistry of structure II

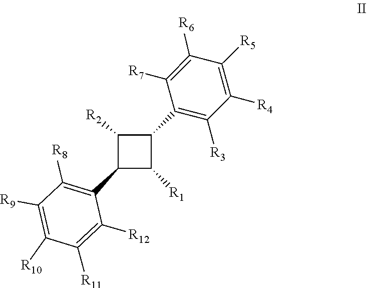

then
R₁ and R₂ are different and are each —C(═O)R₁₃, —C(═O)OR₁₃, —C(═O)O-alkyl-R₁₃, —C(═O)NR₁₃R₁₄, -alkyl-C(═O)R₁₃, -alkyl-C(═O)OR₁₃, -alkyl-C(═O)NR₁₃R₁₄, -alkyl-OC(═O)OR₁₃, -alkyl-OC(═O)R₁₃, -alkyl-OR₁₃, -alkyl-NR₁₃R₁₄, -alkyl-NHC(═O)R₁₃, -alkyl-NHC(═O)OR₁₃, -alkyl-NHC(═O)R₁₃, -alkyl-NHC(═O)NR₁₃R₁₄, -alkyl-NHC(═S)NR₁₃R₁₄, -alkyl-NHC(═NR₁₃)NR₁₃R₁₄, —C(—OH) C(═O)OR₁₃, —C(═O)C(═O)OR₁₃ or —C═C—R₁₃,
wherein R₁₃ and R₁₄ are each, independently, H, CF₃, C₁₋₁₀ alkyl, C₂₋₁₀ alkenyl, C₂₋₁₃ alkynyl, heteroalkyl, cycloalkyl, cycloheteroalkyl, aryl, heteroaryl, heterocyclyl or combine to form a cycloalkyl or heterocyclyl;
   R₃, R₄, R₅, R₆, R₇, R₈, R₉, R₁₃, R₁₁ and R₁₂ are each independently, H, halogen, —NO₂, —CN, —NHR₁₅, —NR₁₅R₁₆, —SR₁₅, —SO₂R₁₅, —OR₁₅, —CO₂R₁₅, CF₃, -alkyl-NR₁₅R₁₆, -alkyl-OR₁₅, C₁₋₁₀ alkyl, C₂₋₁₀ alkenyl, C₂₋₁₀ alkynyl, aryl, heteroaryl, or heterocyclyl;
   wherein R₁₅ and R₁₆ are each, independently, H, CF₃, C₁₋₁₀ alkyl, C₂₋₁₀ alkenyl, C₂₋₁₀ alkynyl, heteroalkyl, cycloheteroalkyl, aryl, heteroaryl, or heterocyclyl;
   or an enantiomer or racemate thereof;
   or a pharmaceutically acceptable salt thereof.

In an embodiment, the FABP5 inhibitor has the structure:

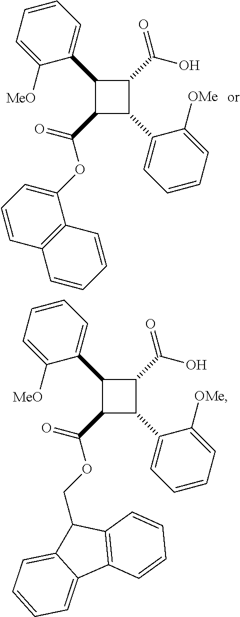

or a pharmaceutically acceptable salt thereof.

In an embodiment, the taxane is paclitaxel, docetaxel, or cabazitaxel. In a preferred embodiment, the taxane is docetaxel or cabazitaxel. In another preferred embodiment, the taxane is docetaxel. In another preferred embodiment, the taxane is cabazitaxel.

In an embodiment, the anticancer therapy is radiation therapy.

In an embodiment, the radiation therapy is external beam radiation. In another embodiment, the radiation therapy is brachytherapy.

In an embodiment, the subject is a mammal.

This invention provides a pharmaceutical composition comprising a FABP5 inhibitor and a pharmaceutically acceptable carrier.

In an embodiment, the pharmaceutical composition of the FABP5 inhibitor has the structure:

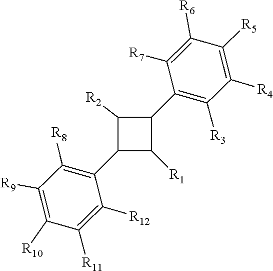

wherein
R₁ and R₂ are different and are each —C(═O)R₁₃, —C(═O)OR₁₃, —C(═O)O-alkyl-R₁₃, —C(═O)NR₁₃R₁₄, -alkyl-C(═O)R₁₃, -alkyl-C(═O)OR₁₃, -alkyl-C(═O)NR₁₃R₁₄, -alkyl-OC(═O)OR₁₃, -alkyl-OC(═O)R₁₃, -alkyl-OR₁₃, -alkyl-NR₁₃R₁₄, -alkyl-NHC(═O)R₁₃, -alkyl-NHC(═O)OR₁₃, -alkyl-NHC(═O)R₁₃, -alkyl-NHC(═O)NR₁₃R₁₄, -alkyl-NHC(═S)NR₁₃R₁₄, -alkyl-NHC(═NR₁₃)NR₁₃R₁₄, —C(—OH) C(═O)OR₁₃, —C(═O)C(═O)OR₁₃ or —C═C—R₁₃,
wherein R₁₃ and R₁₄ are each, independently, H, CF₃, C₁₋₁₀ alkyl, C₂₋₁₀ alkenyl, C₂₋₁₀ alkynyl, heteroalkyl, cycloalkyl, cycloheteroalkyl, aryl, heteroaryl, heterocyclyl or combine to form a cycloalkyl or heterocyclyl;
   R₃, R₄, R₅, R₆, R₇, R₈, R₉, R₁₀, R₁₁ and R₁₂ are each independently, H, halogen, —NO₂, —CN, —NHR₁₅, —NR₁₅R₁₆, —SR₁₅, —SO₂R₁₅, —OR₁₅, —CO₂R₁₅, CF₃, -alkyl-NR₁₅R₁₆, -alkyl-OR₁₅, C₁₋₁₀ alkyl, C₂₋₁₀ alkenyl, C₂₋₁₀ alkynyl, aryl, heteroaryl, or heterocyclyl;
   wherein R₁₅ and R₁₆ are each, independently, H, CF₃, C₁₋₁₀ alkyl, C₂₋₁₀ alkenyl, C₂₋₁₀ alkynyl, heteroalkyl, cycloheteroalkyl, aryl, heteroaryl, or heterocyclyl;

In an embodiment of the above, the compound of the pharmaceutical composition has the stereochemistry of structure I

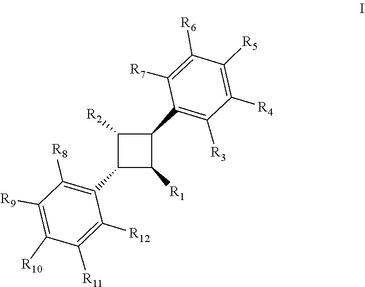

then
R₁ and R₂ are different and are each —C(═O)R₁₃, —C(═O)OR₁₃, —C(═O)O-alkyl-R₁₃, —C(═O)NR₁₃R₁₄, -alkyl-C(═O)R₁₃, -alkyl-C(═O)OR₁₃, -alkyl-C(═O)NR₁₃R₁₄, -alkyl-OC(═O)OR₁₃, -alkyl-OC(═O)R₁₃, -alkyl-NR₁₃R₁₄, -alkyl-NHC(═O)R₁₃, -alkyl-NHC(═O)OR₁₃, -alkyl-NHC(═O)R₁₃, -alkyl-NHC(═O)NR₁₃R₁₄, -alkyl-NHC(═S)NR₁₃R₁₄, -alkyl-NHC(═NR₁₃)NR₁₃R₁₄, —C(—OH) C(═O)OR₁₃, C(═O)C(═O)OR₁₃ or —C═C—R₁₃,
wherein R₁₃ and R₁₄ are each, independently, H, CF₃, C₁₋₁₀ alkyl, C₂₋₁₀ alkenyl, C₂₋₁₀ alkynyl, heteroalkyl, cycloalkyl, cycloheteroalkyl, aryl, heteroaryl, heterocyclyl or combine to form a cycloalkyl or heterocyclyl;
   R₃, R₄, R₅, R₆, R₇, R₈, R₉, R₁₀, R₁₁ and R₁₂ are each independently, H, halogen, —NO₂, —CN, —NHR₁₅, —NR₁₅R₁₆, —SR₁₅, —SO₂R₁₅, —OR₁₅, —CO₂R₁₅, CF₃, -alkyl-NR₁₅R₁₆, -alkyl-OR₁₅, C₁₋₁₀ alkyl, C₂₋₁₀ alkenyl, C₂₋₁₀ alkynyl, aryl, heteroaryl, or heterocyclyl;
   wherein R₁₅ and R₁₆ are each, independently, H, CF₃, C₁₋₁₉ alkyl, C₂₋₁₃ alkenyl, C₂₋₁₀ alkynyl, heteroalkyl, cycloheteroalkyl, aryl, heteroaryl, or heterocyclyl;
   or an enantiomer or racemate thereof;
   or a pharmaceutically acceptable salt thereof.

In an embodiment of the above, the compound of the pharmaceutical composition has the stereochemistry of structure II

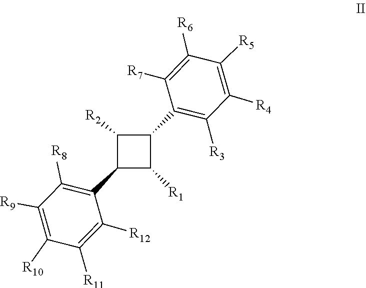

then
R₁ and R₂ are different and are each —C(═O)R₁₃, —C(═O)OR₁₃, —C(═O)O-alkyl-R₁₃, —C(═O)NR₁₃R₁₄, -alkyl-C(═O)R₁₃, -alkyl-C(═O)OR₁₃, -alkyl-C(═O)NR₁₃R₁₄, -alkyl-OC(═O)OR₁₃, -alkyl-OC(═O)R₁₃, -alkyl-OR₁₃, -alkyl-NR₁₃R₁₄, -alkyl-NHC(═O)R₁₃, -alkyl-NHC(═O)OR₁₃, -alkyl-NHC(═O)R₁₃, -alkyl-NHC(═O)NR₁₃R₁₄, -alkyl-NHC(═S)NR₁₃R₁₄, -alkyl-NHC(═NR₁₃)NR₁₃R₁₄, —C(—OH) C(═O)OR₁₃, —C(═O)C(═O)OR₁₃ or —C═C—R₁₃,
wherein R₁₃ and R₁₄ are each, independently, H, CF₃, C₁₋₁₀ alkyl, C₂₋₁₀ alkenyl, C₂₋₁₃ alkynyl, heteroalkyl, cycloalkyl, cycloheteroalkyl, aryl, heteroaryl, heterocyclyl or combine to form a cycloalkyl or heterocyclyl;
   R₃, R₄, R₅, R₆, R₇, R₈, R₉, R₁₃, R₁₁ and R₁₂ are each independently, H, halogen, —NO₂, —CN, —NHR₁₅, —NR₁₅R₁₆, —SR₁₅, —SO₂R₁₅, —OR₁₅, —CO₂R₁₅, CF₃, -alkyl-NR₁₅R₁₆, -alkyl-OR₁₅, C₁₋₁₀ alkyl, C₂₋₁₀ alkenyl, C₂₋₁₀ alkynyl, aryl, heteroaryl, or heterocyclyl;
   wherein R₁₅ and R₁₆ are each, independently, H, CF₃, C₁₋₁₀ alkyl, C₂₋₁₀ alkenyl, C₂₋₁₀ alkynyl, heteroalkyl, cycloheteroalkyl, aryl, heteroaryl, or heterocyclyl;
   or an enantiomer or racemate thereof;
   or a pharmaceutically acceptable salt thereof.

In an embodiment, the pharmaceutical composition of the FABP5 inhibitor has the structure:

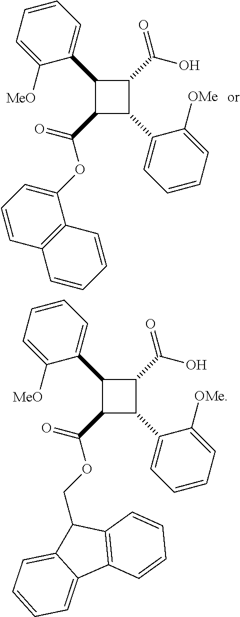

In an embodiment of the above, the pharmaceutical composition further comprises a taxane.

In an embodiment of the above, the pharmaceutical composition comprises docetaxel. In another embodiment of the above, the pharmaceutical composition comprises cabazitaxel.

This invention also provides the use of a FABP5 inhibitor in combination or as an add on with an anticancer therapy in treating a subject afflicted with cancer, wherein the FABP5 inhibitor and the anticancer therapy are administered simultaneously, contemporaneously or concomitantly.

In an embodiment of the above, the FABP5 inhibitor has the structure:

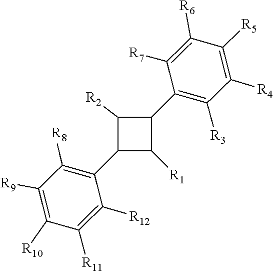

wherein

R₁ and R₂ are different and are each —C(═O)R₁₃, —C(═O)OR₁₃, —C(═O)O-alkyl-R₁₃, —C(═O)NR₁₃R₁₄, -alkyl-C(═O)R₁₃, -alkyl-C(═O)OR₁₃, -alkyl-C(═O)NR₁₃R₁₄, -alkyl-OC(═O)OR₁₃, -alkyl-OC(═O)R₁₃, -alkyl-OR₁₃, -alkyl-NR₁₃R₁₄, -alkyl-NHC(═O)R₁₃, -alkyl-NHC(═O)OR₁₃, -alkyl-NHC(═O)R₁₃, -alkyl-NHC(═O)NR₁₃R₁₄, -alkyl-NHC(═S)NR₁₃R₁₄, -alkyl-NHC(═NR₁₃)NR₁₃R₁₄, —C(—OH) C(═O)OR₁₃, —C(═O)C(═O)OR₁₃ or —C═C—R₁₃,
wherein R₁₃ and R₁₄ are each, independently, H, CF₃, C₁₋₁₀ alkyl, C₂₋₁₀ alkenyl, C₂₋₁₀ alkynyl, heteroalkyl, cycloalkyl, cycloheteroalkyl, aryl, heteroaryl, heterocyclyl or combine to form a cycloalkyl or heterocyclyl;
   R₃, R₄, R₅, R₆, R₂, R₈, R₉, R₁₀, R₁₁ and R₁₂ are each independently, H, halogen, —NO₂, —CN, —NHR₁₅, —NR₁₅R₁₆, —SR₁₅, —SO₂R₁₅, —OR₁₅, —CO₂R₁₅, CF₃, -alkyl-NR₁₅R₁₆, -alkyl-OR₁₅, C₁₋₁₀ alkyl, C₂₋₁₀ alkenyl, C₂₋₁₀ alkynyl, aryl, heteroaryl, or heterocyclyl;
   wherein R₁₅ and R₁₆ are each, independently, H, CF₃, C₁₋₁₀ alkyl, C₂₋₁₀ alkenyl, C₂₋₁₀ alkynyl, heteroalkyl, cycloheteroalkyl, aryl, heteroaryl, or heterocyclyl;
   or an enantiomer or racemate thereof;
   or a pharmaceutically acceptable salt thereof.

In an embodiment of the above, the anticancer therapy is a taxane.

In an embodiment of the above, the anticancer therapy is radiation therapy.

In an embodiment of the above, the cancer is prostate cancer.

This invention also provides the use of a FABP5 inhibitor in the manufacturing of a medicament for use in combination or as an add on with an anticancer therapy in treating a subject afflicted with cancer, wherein the FABP5 inhibitor and the anticancer therapy are administered simultaneously, contemporaneously or concomitantly.

This invention also provides a pharmaceutical composition comprising an amount of a FABP5 inhibitor and an amount of an anticancer therapy for use in treating a subject afflicted with cancer, wherein the FABP5 inhibitor and the anticancer therapy are administered simultaneously, contemporaneously or concomitantly.

In an embodiment of the above, the pharmaceutical composition, wherein the FABP5 inhibitor has the structure:

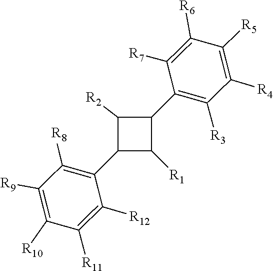

wherein

R₁ and R₂ are different and are each —C(═O)R₁₃, —C(═O)OR₁₃, —C(═O)O-alkyl-R₁₃, —C(═O)NR₁₃R₁₄, -alkyl-C(═O)R₁₃, -alkyl-C(═O)OR₁₃, -alkyl-C(═O)NR₁₃R₁₄, -alkyl-OC(═O)OR₁₃, -alkyl-OC(═O)R₁₃, -alkyl-OR₁₃, -alkyl-NR₁₃R₁₄, -alkyl-NHC(═O)R₁₃, -alkyl-NHC(═O)OR₁₃, -alkyl-NHC(═O)R₁₃, -alkyl-NHC(═O)NR₁₃R₁₄, -alkyl-NHC(═S)NR₁₃R₁₄, -alkyl-NHC(═NR₁₃)NR₁₃R₁₄, —C(—OH) C(═O)OR₁₃, —C(═O)C(═O)OR₁₃ or —C═C—R₁₃,
wherein R₁₃ and R₁₄ are each, independently, H, CF₃, C₁₋₁₀ alkyl, C₂₋₁₀ alkenyl, C₂₋₁₀ alkynyl, heteroalkyl, cycloalkyl, cycloheteroalkyl, aryl, heteroaryl, heterocyclyl or combine to form a cycloalkyl or heterocyclyl;
   R₃, R₄, R₅, R₆, R₇, R₈, R₉, R₁₀, R₁₁ and R₁₂ are each independently, H, halogen, —NO₂, —CN, —NHR₁₅, —NR₁₅R₁₆, —SR₁₅, —SO₂R₁₅, —OR₁₅, —CO₂R₁₅, CF₃, -alkyl-NR₁₅R₁₆, -alkyl-OR₁₅, C₁₋₁₀ alkyl, C₂₋₁₀ alkenyl, C₂₋₁₀ alkynyl, aryl, heteroaryl, or heterocyclyl;
   wherein R₁₅ and R₁₆ are each, independently, H, CF₃, C₁₋₁₀ alkyl, C₂₋₁₀ alkenyl, C₂₋₁₀ alkynyl, heteroalkyl, cycloheteroalkyl, aryl, heteroaryl, or heterocyclyl;
   or an enantiomer or racemate thereof;
   or a pharmaceutically acceptable salt thereof.

In an embodiment of the above, the pharmaceutical composition, wherein the anticancer therapy is a taxane.

In an embodiment of the above, the pharmaceutical composition, wherein the anticancer therapy is radiation therapy.

In an embodiment of the above, the pharmaceutical composition, wherein the cancer is prostate cancer.

Another embodiment relates to a method for treating or preventing cancer, comprising administering to a patient in need thereof a therapeutically effective amount of a FABP5 inhibitor in combination with a taxane. Another embodiment relates to a method for treating or preventing cancer comprising administering to a patient in need thereof a therapeutically effective amount of a pharmaceutical composition comprising a FABP5 inhibitor and a taxane. Another embodiment relates to a method for treating cancer, comprising administering to a patient in need thereof a therapeutically effective amount of SBFI-102 in combination with a taxane. Another embodiment relates to a method for treating cancer, comprising administering to a patient in need thereof a therapeutically effective amount of a pharmaceutical composition comprising SBFI-102 and a taxane. Another embodiment relates to a method for treating cancer, comprising administering to a patient in need thereof a therapeutically effective amount of SBFI-103 in combination with a taxane. Another embodiment relates to a method for treating cancer, comprising administering to a patient in need thereof a therapeutically effective amount of a pharmaceutical composition comprising SBFI-103 and a taxane.

In certain embodiments, a method of treating cancer includes administering a therapeutically effective amount of an FABP5 inhibitor in combination with a taxane to a patient in need thereof.

In certain embodiments, a method of treating cancer includes administering a therapeutically effective amount of SBFI-102 or SBFI-103 in combination with a taxane to a patient in need thereof.

In certain embodiments, a method of treating cancer includes administering a therapeutically effective amount of an FABP5 inhibitor in combination with docetaxel or cabazitaxel to a patient in need thereof.

In certain embodiments, a method of treating cancer includes administering a therapeutically effective amount of SBFI-102 or SBFI-103 in combination with docetaxel or cabazitaxel to a patient in need thereof.

In an embodiment of the above, the combination of a FABP5 inhibitor with a taxane results in a synergistic anticancer effect. In an embodiment of the above, the combination of a FABP5 inhibitor with a taxane results in an additive anticancer effect.

According to further embodiments, the above methods are used in treating prostate cancer. According to further embodiments, the above methods are used in treating drug-resistant prostate cancer.

Taxanes, such as docetaxel and cabazitaxel are utilized in standard treatment regimens for chemotherapy naïve castration-resistant prostate cancer. However, tumors often develop resistance to taxane chemotherapeutics. In an embodiment, the combination of a FABP5 inhibitor with a taxane results in the delay of resistance to taxane chemotherapeutics. In another embodiment of the above, the combination prevents the development of resistance to taxane chemotherapeutics. In an embodiment, the combination of a FABP5 inhibitor with a taxane results in the delay of resistance to FABP5 inhibitors. In another embodiment of the above, the combination prevents the development of resistance to FABP5 inhibitors.

In an embodiment, the combination of a FABP5 inhibitor with a taxane enables use of lower taxane doses than would be required when used alone. In an embodiment of the above, the combination results in reduced adverse effects associated with taxane therapy. In an embodiment, the combination of a FABP5 inhibitor with a taxane enables use of lower FABP5 inhibitor doses than would be required when used alone. In an embodiment of the above, the combination results in reduced adverse effects associated with FABP5 inhibitor therapy.

In an embodiment, the anticancer activity of a FABP5 inhibitor is synergistic with a taxane.

In an embodiment, the cancer displays enhanced expression of FABP5.

Examples of cancers that overexpress FABP5 include, but are not limited to, prostate cancer, skin cancer, breast cancer, hepatocellular carcinoma and cervical cancer.

In one embodiment, the compounds described herein inhibit FABP5. In other embodiments, compounds of the herein described subject matter may be radiolabeled.

In an embodiment, combination therapy includes a taxane with a FAB5 inhibitor. In a preferred embodiment, the taxane is docetaxel. In another preferred embodiment, the taxane is cabazitaxel.

In one embodiment, the FABP5 inhibitor and the anticancer therapy are administered concurrently. In another embodiment, the FABP5 inhibitor and the anticancer therapy are administered sequentially.

In one embodiment, the FABP5 inhibitor and the anticancer therapy are administered simultaneously. In one embodiment, the FABP5 inhibitor and the anticancer therapy are administered contemporaneously. In one embodiment, the FABP5 inhibitor and the anticancer therapy are administered concomitantly.

In one embodiment, the FABP5 inhibitor is SBFI-102. “SBFI-102” has the structure:

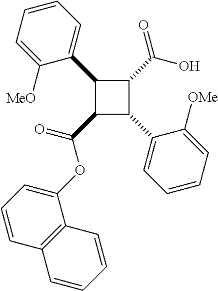

In one embodiment, the FABP5 inhibitor is SBFI-103 “SBFI-103” has the structure:

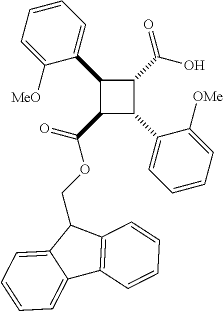

In one embodiment, the combination of an FABP5 inhibitor with a taxane results in enhanced antitumor efficacy in a subject. In one embodiment, the combination of an FABP5 inhibitor with a taxane results in synergistic antitumor efficacy in a subject.

In another embodiment, the combination of an FABP5 inhibitor with a taxane enables the use of lower taxane doses in a subject.

In another embodiment, the combination of an FABP5 inhibitor with a taxane reduces resistance to the effects of a taxane.

In another embodiment, the combination of an FABP5 inhibitor with a taxane decreases the adverse effects associated with taxane chemotherapeutics.

In one embodiment, FABP5 inhibitors potentiate the cytotoxic and tumor-suppressive effects of docetaxel or cabazitaxel.

In one embodiment, a FABP5 inhibitor is administered in combination with radiation therapy.

In an embodiment of the above, the FABP5 inhibitor is SBFI-102.

In an embodiment of the above, the FABP5 inhibitor is SBFI-103.

In a preferred embodiment of the above, the radiation therapy is external beam therapy.

In a preferred embodiment of the above, the radiation therapy is brachytherapy.

In one embodiment, the FABP5 inhibitor is radiolabeled. In another embodiment, SBFI-102 is radiolabeled. In another embodiment, SBFI-103 is radiolabeled.

In an embodiment of the above, the FABP5 inhibitor is radiolabeled with carbon-11. In another embodiment, the FABP5 inhibitor is radiolabeled with nitrogen-13. In another embodiment, the FABP5 inhibitor is radiolabeled with oxygen-15. In another embodiment, the FABP5 inhibitor is radiolabeled with fluorine-18. In another embodiment, the FABP5 inhibitor is radiolabeled with gallium-68. In another embodiment, the FABP5 inhibitor is radiolabeled with zirconium-89. In another embodiment, the FABP5 inhibitor is radiolabeled with rubidium-82. In another embodiment, the FABP5 inhibitor is radiolabeled with copper-64.

In another embodiment, the FABP5 inhibitor is radiolabeled with yttrium-86. In another embodiment, the FABP5 inhibitor is radiolabeled with bromine-76. In another embodiment, the FABP5 inhibitor is radiolabeled with iodine-123. In another embodiment, the FABP5 inhibitor is radiolabeled with iodine-124. In another embodiment, the FABP5 inhibitor is radiolabeled with technetium-99. In another embodiment, the FABP5 inhibitor is radiolabeled with xenon-133. In another embodiment, the FABP5 inhibitor is radiolabeled with thallium-201.

In another embodiment, provides methods for radiolabeling FABP5 inhibitors. In another embodiment, provides methods for radiolabeling SBFI-102. In another embodiment, provides methods for radiolabeling SBFI-103.

The quantity of active component in a unit dose preparation may be varied or adjusted from 0.1 mg to 10000 mg, more typically 1.0 mg to 1000 mg, most typically 10 mg to 500 mg, according to the particular application and the potency of the active component. The composition can, if desired, also contain other compatible therapeutic agents.

The quantity of FABP5 inhibitor in a unit dose preparation may be varied or adjusted from 0.1 mg to 10000 mg, more typically 1.0 mg to 1000 mg, most typically 10 mg to 500 mg, according to the particular application and the potency of the FABP5 inhibitor. The composition can, if desired, also contain other compatible therapeutic agents.

Docetaxel and cabazitaxel may be used at their approved dose levels. The approved dose levels for docetaxel and cabazitaxel are described in the Physician's Desk Reference (Physicians' Desk Reference, 2017), the entire content of which is hereby incorporated by reference herein.

Any concentration ranges, percentage range, or ratio range recited herein are to be understood to include concentrations, percentages or ratios of any integer within that range and fractions thereof, such as one tenth and one hundredth of an integer, unless otherwise indicated.

### Terms

As used herein, and unless stated otherwise, each of the following terms shall have the definition set forth below.

As used herein, the terms “administer”, “administering”, and “administration”, refer to any method which, in sound medical practice, delivers the composition to a subject in such a manner as to provide a therapeutic effect.

As used herein, the phrases an “effective amount” or a “therapeutically effective amount” of an active agent or ingredient, or pharmaceutically active agent or ingredient, which are synonymous herein, refer to an amount of the pharmaceutically active agent sufficient enough to have a therapeutic effect upon administration. A therapeutically effective amount of the pharmaceutically active agent may, will, or is expected to cause a relief of symptoms. Effective amounts of the pharmaceutically active agent will vary with the particular condition or conditions being treated, the severity of the condition, the duration of the treatment, the specific components of the composition being used, and like factors.

As used herein, “subject” or “individual” or “animal” or “patient” or “mammal”, refers to any subject, particularly a mammalian subject, for whom diagnosis, prognosis, or therapy is desired, for example, a human.

As used herein, a “treatment” or “treating” of a disease, disorder, or condition encompasses alleviation of at least one symptom thereof, a reduction in the severity thereof, or the delay, prevention, or inhibition of the progression thereof. Treatment need not mean that the disease, disorder, or condition is totally cured. A useful composition herein needs only to reduce the severity of a disease, disorder, or condition, reduce the severity of symptoms associated therewith, provide improvement to a patient or subject's quality of life, or delay, prevent, or inhibit the onset of a disease, disorder, or condition.

As used herein, “anticancer therapy” refers to any treatment to stop or prevent cancer. Types of anticancer therapy include but are not limited to chemotherapy, radiation therapy, surgery, immunotherapy.

As used herein, the term “chemotherapy” refers to the use of any agent to treat cancer or that provides a beneficial therapeutic effect to a subject suffering from cancer.

As used herein, the term “radiation therapy” or “radiotherapy” refers to the use of ionizing radiation to control or kill cancer cells. Types of radiation therapy include but are not limited to external beam radiation, brachytherapy, or systemic radioisotope therapy.

Anticancer therapy includes a variety of therapies that are both chemical and radiation based treatments. Chemotherapies include, for example, cisplatin (CDDP), carboplatin, oxaliplatin, irinotecan, topotecan, procarbazine, mechlorethamine, cyclophosphamide, camptothecin, ifosfamide, melphalan, chlorambucil, buSulfan, nitroSurea, dactinomycin, daunorubicin, doxorubicin, bleomycin, plicomycin, mitomycin, etoposide (VP16), tamoxifen, raloxifene, estrogen receptor binding agents, taxane, docetaxel, paclitaxel, Abraxane™, gemcitabine, navelbine, farnesyl-protein transferase inhibitors, transplatinum, 5-fluorouracil, Vincristin, vinblastin, methotrexate, medroxy-progesterone acetate or any analog or derivative variant of the foregoing.

Other examples of chemotherapy include Receptor Tyrosine Kinase Inhibitors (RTKi) which include but are not limited to, Herceptin (Genentech), Laptinib (GSK), Tarceva (Genentech/OSI), Gefitinib (AstraZenca), Fluro-Sorafenib (Bayer), Sorafenib (Bayer), PF-2341066 (Pfizer), or any analog or derivative variant thereof. It is specifically contemplated that any of these compounds or derivatives or analogs, can be used in these combination therapies.

Furthermore, chemotherapy also includes PARP inhibitors, which include but are not limited to 4-(3-(4-cyclopropylcarbonyl)piperazin-4-ylcarbonyl)-4-fluorophenyl)methyl(2H)phthalazin-1-one (Olaparib: AZD2281; KU0059436, AstraZeneca), 2-(2R)-2-methylpyrrolidin-2-yl)-1H-benzimidazole-4-carboxamide (ABT-888, Abbott Laboratories), benzimidazole derivative (ABT-472, Abbott Laboratories), O-(3-piperidino-2-hydroxy-1-propyl)nicotinic amidoxime (BGP15. Allos Therapeutics), AZD2461 (AstraZeneca), BMN673 (BioMarin Pharmaceutical Inc), 3-2-fluoro-5-(4-oxo-3,4-dihydro-phthalazin-1-ylmethyl)-phenyl-5-methyl-imidazolidine-2,4-dione, 3-3-(5,8-difluoro-4-oxo-3,4-dihydro-phthalazin-1-ylmethyl)-phenyl-5-methyl-imidazoline-2,4-dione, 5-chloro-2-1-3-(1,4)diazepane-1-carbonyl)-4-fluoro-phenyl-ethoxy benzamide, 2-3-2-fluoro-5-(4-oxo-3,4-dihydrophthalazin-1-ylmethyl)-phenyl-5-methyl-2, 4-dioxoimidazolidin-1-yl)-acetamide,-4-3-(4-Cyclopropanecarbonyl-piperazine-1-carbonyl)-4-fluorobenzyl-2H-phthalazin-1-one, 3-2-fluoro-5-(4-oxo-3,4,dihydro-phthalazin-1-ylmethyl)-phenyl-5,5-dimethyl-1-(2-(4-methyl-piperazin-1-yl)-2-oxo-ethyl-imidazoline-2,4-dione, 8-fluoro-2-(4-methylaminomethyl-phenyl)-1,3,4,5-tetrahydro-azepino[5.4.3-cd]indol-6-one (WO2008020180), BSI101 (BiPar Sciences), CE9722 (Cephalon Inc), GPI21016 (Eisai Co), PARP Inhibitor ROCHE (F. Hoffman La Roche Ltd), Indoles (INO1001, Genentech), PARP Inhibitors INOTEK (Inotek Pharmaceuticals Co), (S)-2-(4-(piperidin-3-yl)phenyl)-2H-indazole-7-carboxamide hydrochloride (MK4827, Merck & Co), MP124 (Mitsubishi Tanabe Pharma Co), ON02231 (Ono Pharmaceutical Co Ltd), LT673 (LEAD Therapeutics), Indole derivative (PF1367338, Pfizer), 2-quinolinones and 2-quinoxalinones (U.S. Pat. No. 7,879,857), 2-alkyl quinazolinone derivatives (U.S. Pat. No. 7,875,621), 2-pyridone derivatives (U.S. Pat. No. 7,863,280), Pyrrolo[1,2-a]pyrazin-1(2H)-one and pyrrolo[1.2-d][1.2.4]triazin-1 (2H)-one derivatives (U.S. Pat. No. 7,834,015), Thieno2.3-cisoquinolines (U.S. Pat. No. 7,825,129), Phthalazinone derivatives (U.S. Pat. No. 7,092,193), Indenoisoquinolinone (U.S. Pat. No. 7,652,028), 1H-benzimidaZole-4-carboxamides (U.S. Pat. No. 7,595,406), 4-(Substituted aryl)-5-hydroxyisoquinolinone derivative (U.S. Pat. No. 7,425,563), and fused pyridazine derivatives (U.S. Pat. No. 7,402,580).

Radiotherapy can cause DNA damage and includes what are commonly known as γ-rays, X-rays, electron-beam radiation and/or the directed delivery of radioisotopes to tumor cells. Other forms of DNA damaging factors are also contemplated. Such as microwaves and UV-irradiation. It is most likely that all of these factors effect abroad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

As used herein, the term “taxanes” include but are not limited to Paclitaxel (Taxol, Abraxane™), Docetaxel (Taxotere), and Cabazitaxel (Jevtana). Their approved dose levels as well as route of administration is described in the Physician's Desk Reference (Physicians' Desk Reference, 2017), the entire content of which is hereby incorporated by reference herein.

As used herein, the term “fatty acid binding protein” or “FABP” refers to fatty acid binding proteins (FABPs) that function as intracellular carriers that shuttle cannabinoids (and by extension fatty acid amides (FAAs)) to FAAH where cannabinoids are hydrolyzed and degraded. Further, uptake of endocannabinoids (and by extension FAAs) by the cell and the subsequent hydrolysis of endocannabinoids (and by extension FAAs) are enhanced by FABPs, and inhibiting the interaction of endocannabinoids (and by extension FAAs) with FABPs reduces endocannabinoid (and by extension FAA) uptake and hydrolysis. FABPS include, for example, fatty acid binding protein 1 (FABP 1), fatty acid binding protein 2 (FABP 2), fatty acid binding protein 3 (FABP 3), fatty acid binding protein 4 (FABP 4), fatty acid binding protein 5 (FABP 5), fatty acid binding protein 6 (FABP 6), fatty acid binding protein 7 (FABP 7), fatty acid binding protein 8 (FABP 8), fatty acid binding protein 9 (FABP 9), fatty acid binding protein 10 (FABP 10), fatty acid binding protein 11 (FABP 11), fatty acid binding protein 5-like (FABP 5-like 1), fatty acid binding protein 5-like 2 (FABP 5-like 2), fatty acid binding protein 5-like 3 (FABP 5-like 3), fatty acid binding protein 5-like 4 (FABP 5-like 4), fatty acid binding protein 5-like 5 (FABP 5-like 5), fatty acid binding protein 5-like 6 (FABP 5-like 6), and fatty acid binding protein 5-like (FABP 5-like 7) (see Chmurzynska et al. 2006 and PCT International Application Publication No. WO 2010/083532 A1, the contents of each of which are hereby incorporated by reference).

As used herein, the term “endocannabinoid” includes any molecule that activates cannabinoid receptors. Examples of such receptors are CB1 and CB2. Examples of endocannabinoids are arachidonoyl ethanolamide (AEA) and 2-arachidonoyl glycerol (2-AG).

As used herein, the term “FABP5 inhibitors” refers to any molecule that inhibits FABP5. Exemplary FABP5 inhibitors are disclosed in U.S. patent application Ser. Nos. 14/413,621, 16/080,493, US Patent Publications 2015/0183715, 2019/0062261 and U.S. Pat. No. 9,604,904, all of which are incorporated herein by reference.

The term “radiolabel”, as used herein, refers to a moiety comprising a radioactive isotope of at least one element. Exemplary suitable radiolabels include but are not limited to carbon-11, nitrogen-13, oxygen-15, fluorine-18, gallium-68, zirconium-89, rubidium-82, copper-64, yttrium-86, bromine-76, iodine-123, iodine-124, technetium-99, xenon-133, and thallium-201. In some embodiments, a radiolabel is one used in positron emission tomography (PET). In some embodiments, a radiolabel is one used in single-photon emission computed tomography (SPECT).

The term “cancer” refers to a tumor resulting from abnormal or uncontrolled cellular growth.

In certain embodiments, the compounds of the present subject matter are useful in the treatment of cancer. The term “cancer” as used herein includes breast, prostate, lung, colon, stomach, pancreatic, ovarian, brain and hematopoietic cancers, esophageal carcinoma, renal cell carcinoma, bladder cancer, head and neck cancer, leukemias, and sarcomas such as cholangiosarcoma and esophageal sarcoma. In particular, this includes breast and ovarian cancers, prostate cancer, pancreatic cancer, hepatocellular carcinoma, non-small- and small-cell lung cancer (NSCLC and SCLC), colorectal cancer, leukemia, and lymphoma. Included are metastatic cancers, such as, for example, metastatic prostate cancer.

As used herein, the term “therapeutic agent” refers to any agent used to treat a disease or that provides a beneficial therapeutic effect to a subject.

As used herein, the term “activity” refers to the activation, production, expression, synthesis, intercellular effect, and/or pathological or aberrant effect of the referenced molecule, either inside and/or outside of a cell. Such molecules include, but are not limited to, cytokines, enzymes, growth factors, pro-growth factors, active growth factors, and pro-enzymes. Molecules such as cytokines, enzymes, growth factors, pro-growth factors, active growth factors, and pro-enzymes may be produced, expressed, or synthesized within a cell where they may exert an effect. Such molecules may also be transported outside of the cell to the extracellular matrix where they may induce an effect on the extracellular matrix or on a neighboring cell. It is understood that activation of inactive cytokines, enzymes and pro-enzymes may occur inside and/or outside of a cell and that both inactive and active forms may be present at any point inside and/or outside of a cell. It is also understood that cells may possess basal levels of such molecules for normal function and that abnormally high or low levels of such active molecules may lead to pathological or aberrant effects that may be corrected by pharmacological intervention.

In some embodiments, the compounds of the present invention include all hydrates, solvates, and complexes of the compounds used by this invention.

In some embodiments, if a chiral center or another form of an isomeric center is present in a compound of the present invention, all forms of such isomer or isomers, including enantiomers and diastereomers, are intended to be covered herein.

In some embodiments, if a chiral center or another form of an isomeric center is present in a compound of the present invention, only enantiomeric forms are intended to be covered herein.

Compounds containing a chiral center may be used as a racemic mixture, an enantiomerically enriched mixture, or the racemic mixture may be separated using well-known techniques and an individual enantiomer may be used alone. The compounds described in the present invention are in racemic form or as individual enantiomers.

As used herein, “enantiomers” are non-identical, non-superimposable mirror images of each other. For any given chiral compound, only one pair of enantiomers exists. The enantiomers can be separated using known techniques, including those described in Pure and Applied Chemistry 69, 1469-1474, (1997) IUPAC.

In cases in which compounds have unsaturated carbon-carbon double bonds, both the cis (Z) and trans (E) isomers are within the scope of this invention.

The compounds of the subject invention may have spontaneous tautomeric forms. In cases wherein compounds may exist in tautomeric forms, such as keto-enol tautomers, each tautomeric form is contemplated as being included within this invention whether existing in equilibrium or predominantly in one form.

In the compound structures depicted herein, hydrogen atoms are not shown for carbon atoms having less than four bonds to non-hydrogen atoms.

However, it is understood that enough hydrogen atoms exist on said carbon atoms to satisfy the octet rule.

This invention also provides isotopic variants of the compounds disclosed herein, including wherein the isotopic atom is ²H and/or wherein the isotopic atom ¹³C. Accordingly, in the compounds provided herein hydrogen can be enriched in the deuterium isotope. It is to be understood that the invention encompasses all such isotopic forms.

It is understood that the structures described in the embodiments of the methods hereinabove can be the same as the structures of the compounds described hereinabove.

It is understood that where a numerical range is recited herein, the present invention contemplates each integer between, and including, the upper and lower limits, unless otherwise stated.

Except where otherwise specified, if the structure of a compound of this invention includes an asymmetric carbon atom, it is understood that the compound occurs as a racemate, racemic mixture, and isolated single enantiomer. All such isomeric forms of these compounds are expressly included in this invention. Except where otherwise specified, each stereogenic carbon may be of the R or S configuration. It is to be understood accordingly that the isomers arising from such asymmetry (e.g., all enantiomers and diastereomers) are included within the scope of this invention, unless indicated otherwise. Such isomers can be obtained in substantially pure form by classical separation techniques and by stereochemically controlled synthesis, such as those described in “Enantiomers, Racemates and Resolutions” by J. Jacques, A. Collet and

S. Wilen, Pub. John Wiley & Sons, N Y, 1981. For example, the resolution may be carried out by preparative chromatography on a chiral column.

The subject invention is also intended to include all isotopes of atoms occurring on the compounds disclosed herein. Isotopes include those atoms having the same atomic number but different mass numbers. By way of general example and without limitation, isotopes of hydrogen include tritium and deuterium. Isotopes of carbon include C-13 and C-14.

It will be noted that any notation of a carbon in structures throughout this application, when used without further notation, are intended to represent all isotopes of carbon, such as ¹²C, ¹³C, or ¹⁴C. Furthermore, any compounds containing ¹³C or ¹⁴C may specifically have the structure of any of the compounds disclosed herein.

It will also be noted that any notation of a hydrogen in structures throughout this application, when used without further notation, are intended to represent all isotopes of hydrogen, such as ¹H, ²H, or 3H. Furthermore, any compounds containing ²H or ³H may specifically have the structure of any of the compounds disclosed herein.

Isotopically-labeled compounds can generally be prepared by conventional techniques known to those skilled in the art using appropriate isotopically-labeled reagents in place of the non-labeled reagents employed.

In the compounds used in the method of the present invention, the substituents may be substituted or unsubstituted, unless specifically defined otherwise.

In the compounds used in the method of the present invention, alkyl, heteroalkyl, monocycle, bicycle, aryl, heteroaryl and heterocycle groups can be further substituted by replacing one or more hydrogen atoms with alternative non-hydrogen groups. These include, but are not limited to, halo, hydroxy, mercapto, amino, carboxy, cyano, carbamoyl and aminocarbonyl and aminothiocarbonyl.

It is understood that substituents and substitution patterns on the compounds used in the method of the present invention can be selected by one of ordinary skill in the art to provide compounds that are chemically stable and that can be readily synthesized by techniques known in the art from readily available starting materials. If a substituent is itself substituted with more than one group, it is understood that these multiple groups may be on the same carbon or on different carbons, so long as a stable structure results.

In choosing the compounds used in the method of the present invention, one of ordinary skill in the art will recognize that the various substituents, i.e. R₁, R₂, etc. are to be chosen in conformity with well-known principles of chemical structure connectivity.

As used herein, “alkyl” includes both branched and straight-chain saturated aliphatic hydrocarbon groups having the specified number of carbon atoms and may be unsubstituted or substituted. Thus, C₁-Cₙ as in “C₁-Cₙ alkyl” is defined to include individual groups each having 1, 2, . . . , n−1 or n carbons in a linear or branched arrangement. For example, C₁-C₆, as in “C₁-C₆ alkyl” is defined to include individual groups each having 1, 2, 3, 4, 5, or 6 carbons in a linear or branched arrangement, and specifically includes methyl, ethyl, n-propyl, isopropyl, n-butyl, t-butyl, pentyl, hexyl, and octyl.

As used herein, “alkenyl” refers to a non-aromatic hydrocarbon radical, straight or branched, containing at least 1 carbon to carbon double bond, and up to the maximum possible number of non-aromatic carbon-carbon double bonds may be present, and may be unsubstituted or substituted. For example, “C₂-C₆ alkenyl” means an alkenyl radical having 2, 3, 4, 5, or 6 carbon atoms, and up to 1, 2, 3, 4, or 5 carbon-carbon double bonds respectively. Alkenyl groups include ethenyl, propenyl, butenyl and cyclohexenyl.

The term “alkynyl” refers to a hydrocarbon radical straight or branched, containing at least 1 carbon to carbon triple bond, and up to the maximum possible number of non-aromatic carbon-carbon triple bonds may be present, and may be unsubstituted or substituted. Thus, “C₂-C₆ alkynyl” means an alkynyl radical having 2 or 3 carbon atoms and 1 carbon-carbon triple bond, or having 4 or 5 carbon atoms and up to 2 carbon-carbon triple bonds, or having 6 carbon atoms and up to 3 carbon-carbon triple bonds. Alkynyl groups include ethynyl, propynyl and butynyl.

“Alkylene”, “alkenylene” and “alkynylene” shall mean, respectively, a divalent alkane, alkene and alkyne radical, respectively. It is understood that an alkylene, alkenylene, and alkynylene may be straight or branched. An alkylene, alkenylene, and alkynylene may be unsubstituted or substituted.

As used herein, “heteroalkyl” includes both branched and straight-chain saturated aliphatic hydrocarbon groups having the specified number of carbon atoms and at least 1 heteroatom within the chain or branch.

As used herein, “heterocycle” or “heterocyclyl” as used herein is intended to mean a 5- to 10-membered nonaromatic ring containing from 1 to 4 heteroatoms selected from the group consisting of O, N and S, and includes bicyclic groups. “Heterocyclyl” therefore includes, but is not limited to the following: imidazolyl, piperazinyl, piperidinyl, pyrrolidinyl, morpholinyl, thiomorpholinyl, tetrahydropyranyl, dihydropiperidinyl, tetrahydrothiophenyl and the like. If the heterocycle contains a nitrogen, it is understood that the corresponding N-oxides thereof are also encompassed by this definition.

As herein, “cycloalkyl” shall mean cyclic rings of alkanes of three to eight total carbon atoms, or any number within this range (i.e., cyclopropyl, cyclobutyl, cyclopentyl, cyclohexyl, cycloheptyl or cyclooctyl).

As used herein, “monocycle” includes any stable polyatomic carbon ring of up to 10 atoms and may be unsubstituted or substituted. Examples of such non-aromatic monocycle elements include but are not limited to: cyclobutyl, cyclopentyl, cyclohexyl, and cycloheptyl. Examples of such aromatic monocycle elements include but are not limited to: phenyl.

As used herein, “bicycle” includes any stable polyatomic carbon ring of up to 10 atoms that is fused to a polyatomic carbon ring of up to 10 atoms with each ring being independently unsubstituted or substituted. Examples of such non-aromatic bicycle elements include but are not limited to: decahydronaphthalene. Examples of such aromatic bicycle elements include but are not limited to: naphthalene.

As used herein, “aryl” is intended to mean any stable monocyclic, bicyclic or polycyclic carbon ring of up to 10 atoms in each ring, wherein at least one ring is aromatic, and may be unsubstituted or substituted. Examples of such aryl elements include phenyl, p-toluenyl (4-methylphenyl), naphthyl, tetrahydro-naphthyl, indanyl, biphenyl, phenanthryl, anthryl or acenaphthyl. In cases where the aryl substituent is bicyclic and one ring is non-aromatic, it is understood that attachment is via the aromatic ring.

As used herein, the term “polycyclic” refers to unsaturated or partially unsaturated multiple fused ring structures, which may be unsubstituted or substituted.

The term “alkylaryl” refers to alkyl groups as described above wherein one or more bonds to hydrogen contained therein are replaced by a bond to an aryl group as described above. It is understood that an “arylalkyl” group is connected to a core molecule through a bond from the alkyl group and that the aryl group acts as a substituent on the alkyl group. Examples of arylalkyl moieties include, but are not limited to, benzyl (phenylmethyl), p-trifluoromethylbenzyl (4-trifluoromethylphenylmethyl), 1-phenylethyl, 2-phenylethyl, 3-phenylpropyl, 2-phenylpropyl and the like.

The term “heteroaryl”, as used herein, represents a stable monocyclic, bicyclic or polycyclic ring of up to 10 atoms in each ring, wherein at least one ring is aromatic and contains from 1 to 4 heteroatoms selected from the group consisting of O, N and S. Bicyclic aromatic heteroaryl groups include phenyl, pyridine, pyrimidine or pyridizine rings that are (a) fused to a 6-membered aromatic (unsaturated) heterocyclic ring having one nitrogen atom; (b) fused to a 5- or 6-membered aromatic (unsaturated) heterocyclic ring having two nitrogen atoms; (c) fused to a 5-membered aromatic (unsaturated) heterocyclic ring having one nitrogen atom together with either one oxygen or one sulfur atom; or (d) fused to a 5-membered aromatic (unsaturated) heterocyclic ring having one heteroatom selected from O, N or S. Heteroaryl groups within the scope of this definition include but are not limited to: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrazolyl, tetrazolopyridyl, thiadiazolyl, thiazolyl, thienyl, triazolyl, azetidinyl, aziridinyl, 1,4-dioxanyl, hexahydroazepinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, tetrahydrothienyl, acridinyl, carbazolyl, cinnolinyl, quinoxalinyl, pyrrazolyl, indolyl, benzotriazolyl, benzothiazolyl, benzoxazolyl, isoxazolyl, isothiazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetra-hydroquinoline. In cases where the heteroaryl substituent is bicyclic and one ring is non-aromatic or contains no heteroatoms, it is understood that attachment is via the aromatic ring or via the heteroatom containing ring, respectively. If the heteroaryl contains nitrogen atoms, it is understood that the corresponding N-oxides thereof are also encompassed by this definition.

The term “alkylheteroaryl” refers to alkyl groups as described above wherein one or more bonds to hydrogen contained therein are replaced by a bond to an heteroaryl group as described above. It is understood that an “alkylheteroaryl” group is connected to a core molecule through a bond from the alkyl group and that the heteroaryl group acts as a substituent on the alkyl group. Examples of alkylheteroaryl moieties include, but are not limited to, —CH₂—(C₅H₄N), —CH₂—CH₂—(C₅H₄N) and the like.

The term “heterocycle” or “heterocyclyl” refers to a mono- or polycyclic ring system which can be saturated or contains one or more degrees of unsaturation and contains one or more heteroatoms. Preferred heteroatoms include N, O, and/or S, including N-oxides, sulfur oxides, and dioxides. Preferably the ring is three to ten-membered and is either saturated or has one or more degrees of unsaturation. The heterocycle may be unsubstituted or substituted, with multiple degrees of substitution being allowed. Such rings may be optionally fused to one or more of another “heterocyclic” ring(s), heteroaryl ring(s), aryl ring(s), or cycloalkyl ring(s). Examples of heterocycles include, but are not limited to, tetrahydrofuran, pyran, 1,4-dioxane, 1,3-dioxane, piperidine, piperazine, pyrrolidine, morpholine, thiomorpholine, tetrahydrothiopyran, tetrahydrothiophene, 1,3-oxathiolane, and the like.

The alkyl, alkenyl, alkynyl, aryl, heteroaryl and heterocyclyl substituents may be substituted or unsubstituted, unless specifically defined otherwise. In the compounds of the present invention, alkyl, alkenyl, alkynyl, aryl, heterocyclyl and heteroaryl groups can be further substituted by replacing one or more hydrogen atoms with alternative non-hydrogen groups. These include, but are not limited to, halo, hydroxy, mercapto, amino, carboxy, cyano and carbamoyl.

As used herein, the term “halogen” refers to F, Cl, Br, and I.

The terms “substitution”, “substituted” and “substituent” refer to a functional group as described above in which one or more bonds to a hydrogen atom contained therein are replaced by a bond to non-hydrogen or non-carbon atoms, provided that normal valencies are maintained and that the substitution results in a stable compound. Substituted groups also include groups in which one or more bonds to a carbon(s) or hydrogen(s) atom are replaced by one or more bonds, including double or triple bonds, to a heteroatom. Examples of substituent groups include the functional groups described above, and halogens (i.e., F, Cl, Br, and I); alkyl groups, such as methyl, ethyl, n-propyl, isopropryl, n-butyl, tert-butyl, and trifluoromethyl; hydroxyl; alkoxy groups, such as methoxy, ethoxy, n-propoxy, and isopropoxy; aryloxy groups, such as phenoxy; arylalkyloxy, such as benzyloxy (phenylmethoxy) and p-trifluoromethylbenzyloxy (4-trifluoromethylphenylmethoxy); heteroaryloxy groups; sulfonyl groups, such as trifluoromethanesulfonyl, methanesulfonyl, and p-toluenesulfonyl; nitro, nitrosyl; mercapto; sulfanyl groups, such as methylsulfanyl, ethylsulfanyl and propylsulfanyl; cyano; amino groups, such as amino, methylamino, dimethylamino, ethylamino, and diethylamino; and carboxyl. Where multiple substituent moieties are disclosed or claimed, the substituted compound can be independently substituted by one or more of the disclosed or claimed substituent moieties, singly or pluraly. By independently substituted, it is meant that the (two or more) substituents can be the same or different.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

In choosing the compounds of the present invention, one of ordinary skill in the art will recognize that the various substituents, i.e. R₁, R₂, etc. are to be chosen in conformity with well-known principles of chemical structure connectivity. The various R groups attached to the aromatic rings of the compounds disclosed herein may be added to the rings by standard procedures, for example those set forth in Advanced Organic Chemistry: Part B: Reaction and Synthesis, Francis Carey and Richard Sundberg, (Springer) 5th ed. Edition. (2007), the content of which is hereby incorporated by reference.

The compounds used in the method of the present invention may be prepared by techniques well known in organic synthesis and familiar to a practitioner ordinarily skilled in the art. However, these may not be the only means by which to synthesize or obtain the desired compounds.

The compounds used in the method of the present invention may be prepared by techniques described in Vogel's Textbook of Practical Organic Chemistry, A. I. Vogel, A. R. Tatchell, B. S. Furnis, A. J. Hannaford, P. W. G. Smith, (Prentice Hall) 5th Edition (1996), March's Advanced Organic Chemistry: Reactions, Mechanisms, and Structure, Michael B. Smith, Jerry March, (Wiley-Interscience) 5th Edition (2007), and references therein, which are incorporated by reference herein. However, these may not be the only means by which to synthesize or obtain the desired compounds.

Another aspect of the invention comprises a compound used in the method of the present invention as a pharmaceutical composition.

In some embodiments, a pharmaceutical composition comprising the compound of the present invention and a pharmaceutically acceptable carrier.

As used herein, the term “pharmaceutically active agent” means any substance or compound suitable for administration to a subject and furnishes biological activity or other direct effect in the treatment, cure, mitigation, diagnosis, or prevention of disease, or affects the structure or any function of the subject. Pharmaceutically active agents include, but are not limited to, substances and compounds described in the Physicians' Desk Reference (PDR Network, LLC; 64th edition; Nov. 15, 2009) and “Approved Drug Products with Therapeutic Equivalence Evaluations” (U.S. Department Of Health And Human Services, 30th edition, 2010), which are hereby incorporated by reference. Pharmaceutically active agents which have pendant carboxylic acid groups may be modified in accordance with the present invention using standard esterification reactions and methods readily available and known to those having ordinary skill in the art of chemical synthesis. Where a pharmaceutically active agent does not possess a carboxylic acid group, the ordinarily skilled artisan will be able to design and incorporate a carboxylic acid group into the pharmaceutically active agent where esterification may subsequently be carried out so long as the modification does not interfere with the pharmaceutically active agent's biological activity or effect.

The compounds used in the method of the present invention may be in a salt form. As used herein, a “salt” is a salt of the instant compounds which has been modified by making acid or base salts of the compounds. In the case of compounds used to treat an infection or disease caused by a pathogen, the salt is pharmaceutically acceptable. Examples of pharmaceutically acceptable salts include, but are not limited to, mineral or organic acid salts of basic residues such as amines; alkali or organic salts of acidic residues such as phenols. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the alkaline earth metal salts, sodium, potassium or lithium. The term “pharmaceutically acceptable salt” in this respect, refers to the relatively non-toxic, inorganic and organic acid or base addition salts of compounds of the present invention. These salts can be prepared in situ during the final isolation and purification of the compounds of the invention, or by separately reacting a purified compound of the invention in its free base or free acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

The compounds of the present invention may also form salts with basic amino acids such a lysine, arginine, etc. and with basic sugars such as N-methylglucamine, 2-amino-2-deoxyglucose, etc. and any other physiologically non-toxic basic substance.

The compounds used in the method of the present invention may be administered in various forms, including those detailed herein. The treatment with the compound may be a component of a combination therapy or an adjunct therapy, i.e. the subject or patient in need of the drug is treated or given another drug for the disease in conjunction with one or more of the instant compounds. This combination therapy can be sequential therapy where the patient is treated first with one drug and then the other or the two drugs are given simultaneously. These can be administered independently by the same route or by two or more different routes of administration depending on the dosage forms employed.

As used herein, a “pharmaceutically acceptable carrier” is a pharmaceutically acceptable solvent, suspending agent or vehicle, for delivering the instant compounds to the animal or human. The carrier may be liquid or solid and is selected with the planned manner of administration in mind. Liposomes are also a pharmaceutically acceptable carrier as are slow-release vehicles.

The dosage of the compounds administered in treatment will vary depending upon factors such as the pharmacodynamic characteristics of a specific chemotherapeutic agent and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

A dosage unit of the compounds used in the method of the present invention may comprise a single compound or mixtures thereof with additional antitumor agents. The compounds can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by injection, topical application, or other methods, into or topically onto a site of disease or lesion, all using dosage forms well known to those of ordinary skill in the pharmaceutical arts.

The compounds used in the method of the present invention can be administered in admixture with suitable pharmaceutical diluents, extenders, excipients, or in carriers such as the novel programmable sustained-release multi-compartmental nanospheres (collectively referred to herein as a pharmaceutically acceptable carrier) suitably selected with respect to the intended form of administration and as consistent with conventional pharmaceutical practices. The unit will be in a form suitable for oral, nasal, rectal, topical, intravenous or direct injection or parenteral administration. The compounds can be administered alone or mixed with a pharmaceutically acceptable carrier. This carrier can be a solid or liquid, and the type of carrier is generally chosen based on the type of administration being used. The active agent can be co-administered in the form of a tablet or capsule, liposome, as an agglomerated powder or in a liquid form. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

Techniques and compositions for making dosage forms useful in the present invention are described in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol. 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol 40 (Gilbert S. Banker, Christopher T. Rhodes, Eds.). All of the aforementioned publications are incorporated by reference herein.

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds used in the method of the present invention may also be administered in the form of liposome delivery systems, such as small unilamellar vesicles, large unilamellar vesicles, and multilamellar vesicles. Liposomes can be formed from a variety of phospholipids such as lecithin, sphingomyelin, proteolipids, protein-encapsulated vesicles or from cholesterol, stearylamine, or phosphatidylcholines. The compounds may be administered as components of tissue-targeted emulsions.

The compounds used in the method of the present invention may also be coupled to soluble polymers as targetable drug carriers or as a prodrug. Such polymers include polyvinylpyrrolidone, pyran copolymer, polyhydroxylpropylmethacrylamide-phenol, polyhydroxyethylasparta-midephenol, or polyethyleneoxide-polylysine substituted with palmitoyl residues. Furthermore, the compounds may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Gelatin capsules may contain the active ingredient compounds and powdered carriers, such as lactose, starch, cellulose derivatives, magnesium stearate, stearic acid, and the like. Similar diluents can be used to make compressed tablets. Both tablets and capsules can be manufactured as immediate release products or as sustained release products to provide for continuous release of medication over a period of hours. Compressed tablets can be sugar-coated or film-coated to mask any unpleasant taste and protect the tablet from the atmosphere, or enteric coated for selective disintegration in the gastrointestinal tract.

For oral administration in liquid dosage form, the oral drug components are combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

Liquid dosage forms for oral administration can contain coloring and flavoring to increase patient acceptance. In general, water, asuitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, Mack Publishing Company, a standard reference text in this field.

The compounds used in the method of the present invention may also be administered in intranasal form via use of suitable intranasal vehicles, or via transdermal routes, using those forms of transdermal skin patches well known to those of ordinary skill in that art. To be administered in the form of a transdermal delivery system, the dosage administration will generally be continuous rather than intermittent throughout the dosage regimen.

Parenteral and intravenous forms may also include minerals and other materials such as solutol and/or ethanol to make them compatible with the type of injection or delivery system chosen.

The compounds and compositions of the present invention can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by topical administration, injection or other methods, to the afflicted area, such as a wound, including ulcers of the skin, all using dosage forms well known to those of ordinary skill in the pharmaceutical arts.

Specific examples of pharmaceutically acceptable carriers and excipients that may be used to formulate oral dosage forms of the present invention are described in U.S. Pat. No. 3,903,297 to Robert, issued Sep. 2, 1975. Techniques and compositions for making dosage forms useful in the present invention are described-in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol 40 (Gilbert S. Banker, Christopher T. Rhodes, Eds.). All of the aforementioned publications are incorporated by reference herein.

The term “prodrug” as used herein refers to any compound that when administered to a biological system generates the compound of the invention, as a result of spontaneous chemical reaction(s), enzyme catalyzed chemical reaction(s), photolysis, and/or metabolic chemical reaction(s). A prodrug is thus a covalently modified analog or latent form of a compound of the invention.

The active ingredient can be administered orally in solid dosage forms, such as capsules, tablets, powders, and chewing gum; or in liquid dosage forms, such as elixirs, syrups, and suspensions, including, but not limited to, mouthwash and toothpaste. It can also be administered parentally, in sterile liquid dosage forms.

Solid dosage forms, such as capsules and tablets, may be enteric-coated to prevent release of the active ingredient compounds before they reach the small intestine. Materials that may be used as enteric coatings include, but are not limited to, sugars, fatty acids, proteinaceous substances such as gelatin, waxes, shellac, cellulose acetate phthalate (CAP), methyl acrylate-methacrylic acid copolymers, cellulose acetate succinate, hydroxy propyl methyl cellulose phthalate, hydroxy propyl methyl cellulose acetate succinate (hypromellose acetate succinate), polyvinyl acetate phthalate (PVAP), and methyl methacrylate-methacrylic acid copolymers.

The compounds and compositions of the invention can be coated onto stents for temporary or permanent implantation into the cardiovascular system of a subject.

Each embodiment disclosed herein is contemplated as being applicable to each of the other disclosed embodiments. Thus, all combinations of the various elements described herein are within the scope of the invention.

This invention will be better understood by reference to the Experimental Details which follow, but those skilled in the art will readily appreciate that the specific experiments detailed are only illustrative of the invention as described more fully in the claims which follow thereafter.

### EXPERIMENTAL DETAILS

### Example 1. In Vitro Synergistic Anticancer Activity Through Combination of a Taxane and FABP5 Inhibitors

Materials and Methods

Cell-Lines

PC3 cells were obtained from American Type Culture Collection (ATCC; CRL-1435; Manassas, Va.) and were authenticated by the ATCC human short tandem repeat profiling cell authentication service. DU-145 and 22Rv1 cells were also obtained from ATCC (HTB-81 and CRL-2505, respectively; ATCC). PC3, DU-145, and 22Rv1 cell-lines were each grown in Roswell Park Memorial Institute 1640 (RPMI 1640) (Gibco-Thermo Fisher Scientific, Gaithersburg Md.) supplemented with 10% fetal bovine serum (FBS) (Gemini Bio-Products, West Sacramento, Calif.) and 100 units/mL of penicillin/streptomycin (Gibco-Thermo Fisher Scientific) in a humidified incubator containing 95% air and 5% CO2. WI-38 cells were obtained from ATCC (CCL-75). WI-38 cells were grown in Dulbecco's modified Eagle's medium (DMEM) (Gibco-Thermo Fisher Scientific) supplemented with 10% FBS and 100 units/mL of penicillin/streptomycin in a humidified incubator containing 95% air and 5% CO2. RWPE-1 cells were purchased from ATCC (CRL-11609). RWPE-1 cells were grown in keratinocyte serum-free media (K-SFM) (Gibco-Thermo Fisher Scientific) supplemented with 25 mg of bovine pituitary extract (BPE), lmg of recombinant human epidermal growth factor (EGF), and 100 units/mL of penicillin/streptomycin in a humidified incubator containing 95% air and 5% CO2.

Drugs

SBFI-102 and SBFI-103 were synthesized as described (Yan, S. et al. 2018). Docetaxel was obtained from Sigma-Aldrich (St. Louis, Mo.). Cabazitaxel was a gift from the Discovery Chemistry Laboratory (Institute of Chemical Biology and Drug Discovery, Stony Brook University, Stony Brook, N.Y.).

Cytotoxicity Assays

Cytotoxicity of SBFI-102, SBFI-103, docetaxel, and cabazitaxel (both individually, and in combination) were determined using the 3-(4,5-dimethylthiazol-2-yl)-2,5 diphenyl tetrazolium bromide (MTT) colorimetric assay (Sigma-Aldrich). PC3 (2500 cells/well), DU-145, 22Rv1, WI-38 (5000 cells/well), and RWPE-1 (10000 cells/well) cells were seeded into 96-well plates and incubated for 24 hours at 37° C. in their respective media (PC3/DU-145/22Rv1 cells utilized RPMI 1640; WI-38 cells utilized DMEM; RWPE-1 cells utilized K-SFM). PC3, DU-145, and 22Rv1 cells were treated with RPMI 1640 supplemented with 1% FBS containing 0.1 μM to 100 μM SBFI-102 or SBFI-103, and/or 0.003 nM to 300 nM docetaxel or cabazitaxel (both individually, or in combination with SBFI-102 or SBFI-103). WI-38 cells were treated with DMEM supplemented with 1% FBS containing 0.1 μM to 100 μM SBFI-102 or SBFI-103. RWPE-1 cells were treated with K-SFM supplemented with 25 mg of BPE and 1 mg of recombinant human EGF containing 0.1 μM to 100 μM SBFI-102 or SBFI-103. All drugs for in vitro experimentation were dissolved in a vehicle of DMSO at a final concentration of 0.1%. Additionally, the appropriate treatment media for each cell-line supplemented with 0.1% DMSO or 1% sodium dodecyl sulfate was used as either a positive or negative control, respectively. After a 72-hour incubation period, cells were washed with PBS and treated with MTT (0.5 mg/mL in serum-free RPMI 1640, serum-free DMEM, or K-SFM) for 4 hours. The cells were subsequently solubilized using DMSO and the absorbance was read at 562 nm in an F5 Filtermax Multi-Mode Microplate Reader (Molecular Devices, Sunnyvale, Calif.).

Analysis of Combined Drug Effects

Synergism between docetaxel/cabazitaxel and SBFI-102 or SBFI-103 was determined through the combination-index (CI) method using the median-effect principle of mass-action law, derived from Chou and Talalay (Chou, T. C. 2006) using ComboSyn software. Briefly, individual drug concentrations that result in the desired fraction of cells affected (Fa) were measured (ie, the concentration of SBFI-102, SBFI-103, docetaxel, or cabazitaxel, which result in the same fraction of cells killed). The concentration resulting in the desired Fa (eg, Fa=0.5 represents 50% of cells effected) for each drug was plotted on an XYaxis, and a straight line drawn to connect the data points. The coadministration of two drugs that achieves the same desired Fa was then plotted on the same axis. Data points that fall above the line (CI>1) represent antagonism, data points that fall on the line (CI=1) represent an additive interaction, and data points that fall below the line (CI<1) represent synergism.

TABLE 1
Synergy analysis of SBFI-102 or SBFI-103 and docetaxel
combinations in PC3, DU-145, and 22Rv1 cell lines

| | SBFI- | SBFI- | | | | |
|---|---|---|---|---|---|---|
| Cell | 102 | 103 | Docetaxel | Fa | CI | |
| line | (μM) | (μM) | (nM) | value | value | Relationship |
| PC3 | 7.5 | — | 0.03 | 0.786 | 0.897 | Synergistic |
| PC3 | 7.5 | — | 0.3 | 0.950 | 0.009 | Synergistic |
| PC3 | 7.5 | — | 3.0 | 0.992 | 0.160 | Synergistic |
| PC3 | — | 1.0 | 0.03 | 0.402 | 0.710 | Synergistic |
| PC3 | — | 1.0 | 0.3 | 0.432 | 0.889 | Synergistic |
| PC3 | — | 1.0 | 3.0 | 0.700 | 0.394 | Synergistic |
| DU-145 | 7.5 | — | 0.03 | 0.934 | 1.302 | — |
| DU-145 | 7.5 | — | 0.3 | 0.963 | 0.968 | Synergistic |
| DU-145 | 7.5 | — | 3.0 | 0.998 | 0.123 | Synergistic |
| DU-145 | — | 1.0 | 0.03 | 0.516 | 4.473 | — |
| DU-145 | — | 1.0 | 0.3 | 0.676 | 2.697 | — |
| DU-145 | — | 1.0 | 3.0 | 0.935 | 0.445 | Synergistic |
| 22Rv1 | 7.5 | — | 0.03 | 0.795 | 3.846 | — |
| 22Rv1 | 7.5 | — | 0.3 | 0.904 | 0.003 | Synergistic |
| 22Rv1 | 7.5 | — | 3.0 | 0.999 | 0.005 | Synergistic |
| 22Rv1 | — | 1.0 | 0.03 | 0.457 | 1.673 | — |
| 22Rv1 | — | 1.0 | 0.3 | 0.744 | 0.373 | Synergistic |
| 22Rv1 | — | 1.0 | 3.0 | 0.905 | 0.267 | Synergistic |

Abbreviations: Fa, fraction of cells affected; CI, combination-index.

TABLE 2
Synergy analysis of SBFI-102 or SBFI-103 and cabazitaxel
combinations in PC3, DU-145, and 22Rv1 cell lines

| | SBFI- | SBFI- | | | | |
|---|---|---|---|---|---|---|
| Cell- | 102 | 103 | cabazitaxel | Fa | CI | |
| line | (μM) | (μM) | (nM) | value | value | Relationship |
| PC3 | 7.5 | — | 0.03 | 0.998 | 0.0001 | Synergistic |
| PC3 | 7.5 | — | 0.3 | 0.999 | 0.0001 | Synergistic |
| PC3 | 7.5 | — | 3.0 | 0.999 | 0.0001 | Synergistic |
| PC3 | — | 1.0 | 0.03 | 0.379 | 0.899 | Synergistic |
| PC3 | — | 1.0 | 0.3 | 0.420 | 0.942 | Synergistic |
| PC3 | — | 1.0 | 3.0 | 0.853 | 0.373 | Synergistic |
| DU-145 | 7.5 | — | 0.03 | 0.829 | 0.709 | Synergistic |
| DU-145 | 7.5 | — | 0.3 | 0.885 | 0.322 | Synergistic |
| DU-145 | 7.5 | — | 3.0 | 0.939 | 0.136 | Synergistic |
| DU-145 | — | 1.0 | 0.03 | 0.464 | 0.449 | Synergistic |
| DU-145 | — | 1.0 | 0.3 | 0.716 | 0.300 | synergistic |
| DU-145 | — | 1.0 | 3.0 | 0.882 | 0.194 | Synergistic |
| 22Rv1 | 7.5 | — | 0.03 | 0.777 | 0.707 | Synergistic |
| 22Rv1 | 7.5 | — | 0.3 | 0.899 | 0.396 | Synergistic |
| 22Rv1 | 7.5 | — | 3.0 | 0.998 | 0.003 | Synergistic |
| 22Rv1 | — | 1.0 | 0.03 | 0.577 | 0.731 | Synergistic |
| 22Rv1 | — | 1.0 | 0.3 | 0.728 | 0.549 | Synergistic |
| 22Rv1 | — | 1.0 | 3.0 | 0.823 | 0.659 | Synergistic |

Abbreviations: Fa, fraction of cells affected; CI, combination-index.

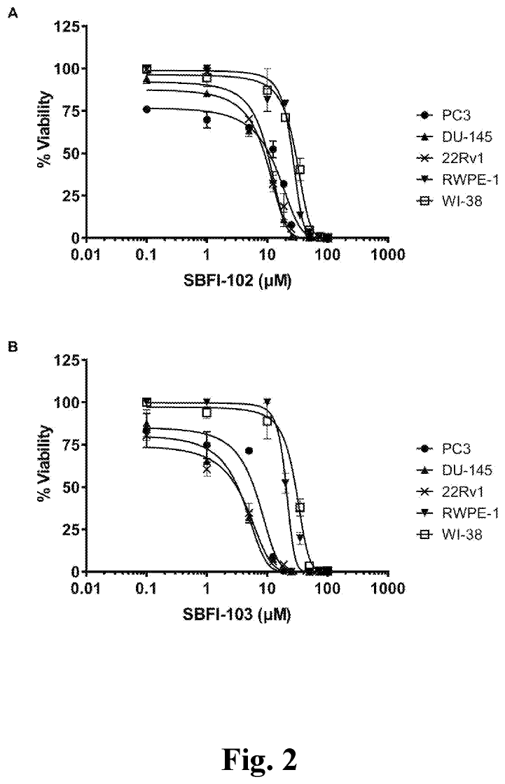

The cytotoxic effects of SBFI-102 (FIG. 2A) and SBFI-103 (FIG. 2B) were assessed in human-derived PC3, DU-145, and 22Rv1 cells that express FABP5 (Kawaguchi, K. et al. 2016). SBFI-102 and SBFI-103 produced dose-dependent cytotoxicity in each cell-line tested: PC3 cells with IC50 values of 11.4 and 6.3 μM, respectively; DU-145 cells with IC50 values of 8.9 and 3.3 μM, respectively; and 22Rv1 cells with IC50 values of 10.1 and 3.1 μM, respectively. Both SBFI-102 and SBFI-103 showed less cytotoxicity in RWPE-1 cells (a normal prostate cell-line), producing IC50 values of 26.0 and 20.6 μM, respectively (FIG. 2A,B). Both SBFI-102 and SBFI-103 showed less cytotoxicity in WI-38 cells (a normal lung cell-line), producing IC50 values of 29.4 and 29.6 μM, respectively (FIG. 2A,B).

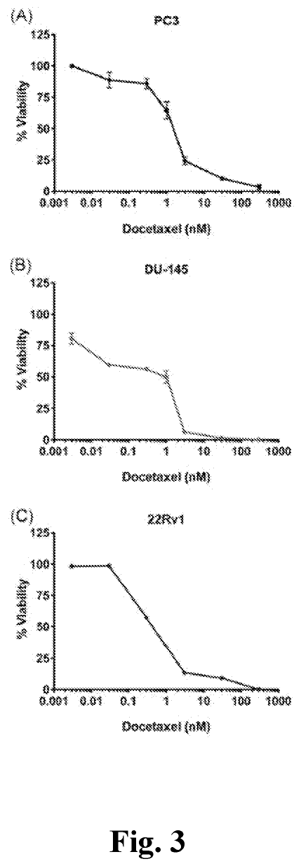

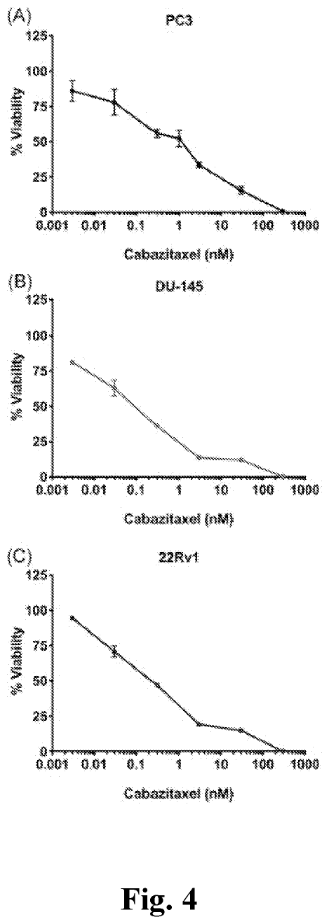

Docetaxel produced dose dependent cytotoxicity in each cell line tested: PC3 cells with an IC50 value of 1.9 nM (FIG. 3A); DU-145 cells with an IC50 value of 0.8 nM (FIG. 3B); and 22Rv1 cells with an IC50 value of 0.3 nM (FIG. 3C). Similarly, cabazitaxel produced dose dependent cytotoxicity in each cell line tested: PC3 cells with an IC50 value of 1.6 nM (FIG. 4A); DU-145 cells with an IC50 value of 0.2 nM (FIG. 4B); and 22Rv1 cells with an IC50 value of 0.3 nM (FIG. 4C).

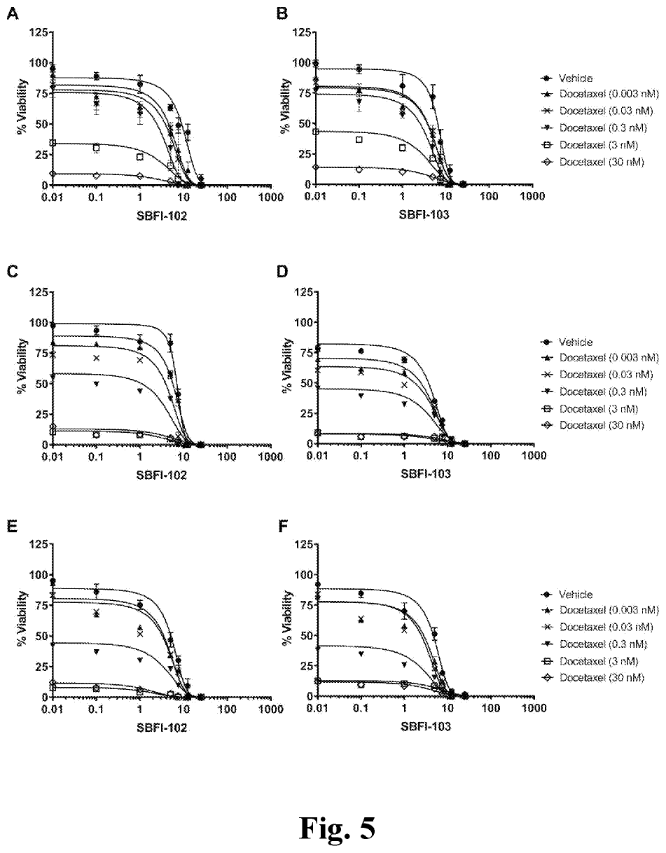

A combination of docetaxel with FABP5 inhibitors SBFI-102 or SBFI-103 resulted in greater cytotoxicity in PC3, DU-145, and 22Rv1 cells than each drug when administered independently (FIG. 5). Synergistic relationships were observed between docetaxel and the FABP5 inhibitors in each cell-line (CI<1) (Table 1).

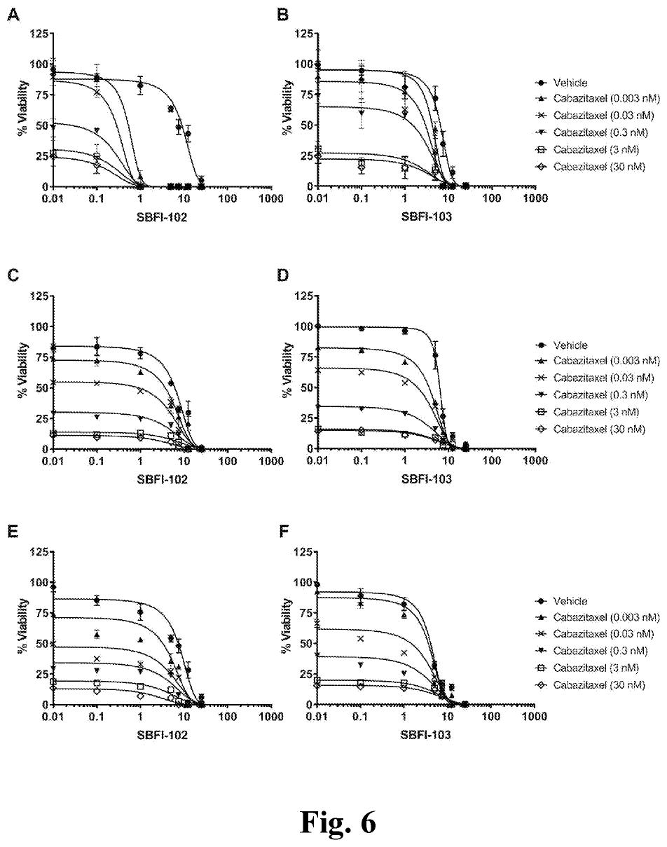

A combination of cabazitaxel with FABP5 inhibitors SBFI-102 or SBFI-103 resulted in greater cytotoxicity in PC3, DU-145, and 22Rv1 cells than each drug when administered independently (FIG. 6). Synergistic relationships between cabazitaxel and the FABP5 inhibitors were also observed (Table 2).

### Example 2. In Vivo Synergistic Anticancer Activity Through Combination of a Taxane and FABP5 Inhibitors

Materials and Methods

Animals

Male BALB/c nude mice (BALB/cOlaHsd-Foxnlnu, 20-30 g, 7-8 weeks old) (Envigo RMS Inc, Indianapolis, Ind.) were used for all experiments. Animals were housed individually at room temperature and were kept on a 12:12-hour light:dark cycle with access to food and water ad libitum. Euthanasia was carried out utilizing CO2 asphyxiation. All of the experiments were approved by the Stony Brook University Animal Care and Use Committee.

Subcutaneous Tumor Implantation

Male BALB/c nude mice were subcutaneously inoculated with PC3 cells. Briefly, cells (1×10⁶ per mouse) were resuspended in 100 μL of a 1:1 mixture of phosphate-buffered saline (PBS):Matrigel (Corning Inc, Corning, N.Y.) and implanted into a single dorsal lateral flank using a 21G needle. Tumor length (L) and tumor width (W) were measured twice weekly using digital calipers, and tumor volume (V) was calculated as (V═[L×W2]/2). When tumor volume reached approximately 150 to 200 mm3, animals were grouped and drug administration commenced. Humane endpoints for all animals were as follows: animals carrying a tumor burden greater than 35 days, body weight (which was recorded twice weekly) decreasing by greater than 15%, tumor ulceration, paralysis, failure to groom, bleeding, respiratory distress, and/or tumor volume reaching 1500 mm3.

Drug Administration

SBFI-102, SBFI-103, and docetaxel were each reconstituted in a 1:1:8 vehicle consisting of dimethyl sulfoxide (DMSO) (Thermo Fisher Scientific, Hampton, N.H.):Cremaphor-EL (Sigma-Aldrich):saline. SBFI-102 and SBFI-103 were administered via intraperitoneal injection (ip) using a 27G needle at 20 mg/kg daily. Docetaxel was administered i.p. at 5 or 10 mg/kg weekly. All drugs were administered in a volume of 10 μL/g body weight.

Quantification and Statistical Analysis

All data were obtained from at least three independent experiments and then values described in each figure legend depict each independent trial or animal. Data for all in vivo experiments were analyzed using a one-way analysis of variance with the Tukey post hoc test (GraphPad Prism, version 8.0.2). Data are represented as means±SEM and P<0.05 was considered statistically significant. The degree of significance is indicated in each figure legend.

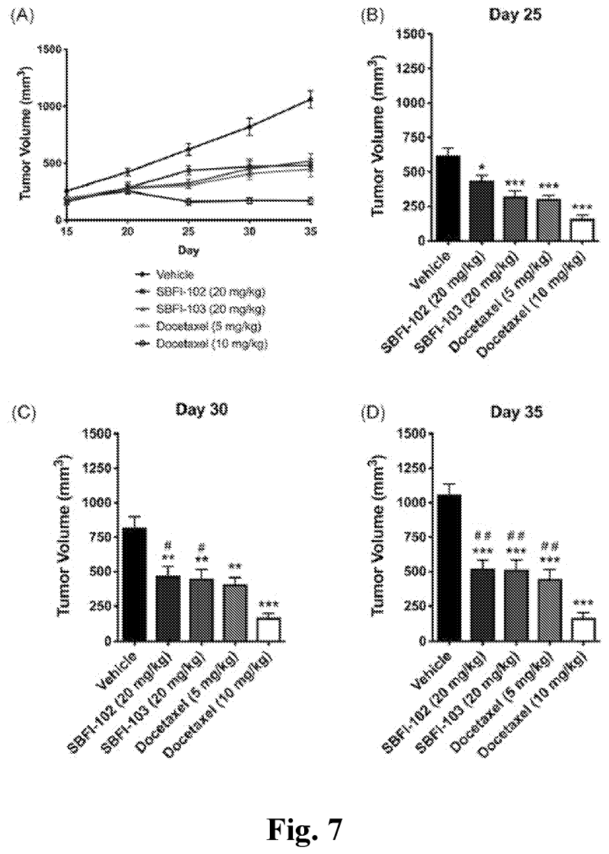

Administration of SBFI-102 or SBFI-103 (20 mg/kg, ip, once daily) significantly reduced tumor growth (FIG. 7A). Similarly, administration of docetaxel (5 or 10 mg/kg, ip, once weekly) reduced tumor growth, with the 5 mg/kg dose producing similar inhibition of tumor growth as observed with the FABP5 inhibitors, while the 10 mg/kg dose produced near complete inhibition of growth (FIG. 7A-D).

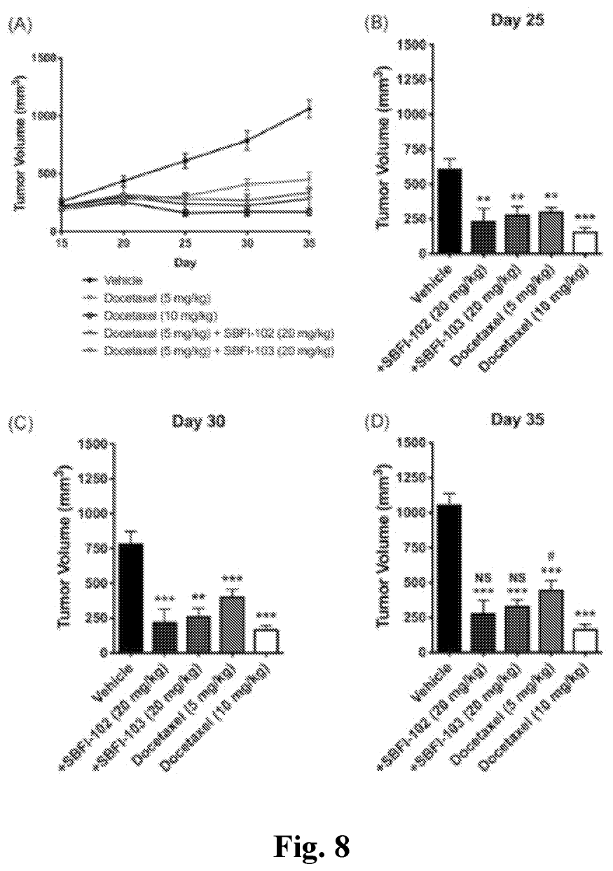

To determine whether SBFI-102 and SBFI-103 enhance the tumor suppressive effects of docetaxel, FABP5 inhibitors were administered in combination with the submaximal dose of docetaxel (5 mg/kg). Consistent with the in vitro efficacy data, coadministration of docetaxel with SBFI-102 or SBFI-103 produced greater inhibition of tumor growth than treatment with each compound alone, with effects that were comparable in magnitude to the 10 mg/kg docetaxel dose (FIG. 8A-D).

### Discussion

Prostate cancer (PCa) remains the second leading cause of cancer related death among men. Taxanes, such as docetaxel and cabazitaxel are used as standard chemotherapeutic treatment regimens for the treatment of naive castration-resistant prostate cancer (Tannock, I. F. et al 2004; Galletti, G. et al. 2017; Antonarakis, E. & Paller, E. S. 2011; de Bono, J. S. 2010; Higano, C. S. & Crawford, E. D. 2011). Despite the clinical availability of docetaxel, cabazitaxel, and newer-generation taxane chemotherapeutics, prostate tumors often develop resistance to these agents (Galletti, G. et al. 2017; Hongo, H. et al. 2018).

Combination therapies consisting of docetaxel/cabazitaxel and other chemotherapeutic agents could lead to enhanced antitumor efficacy or permit the use of lower taxane doses in patients, thus reducing taxane-resistance as well as potentially decreasing the adverse effects associated with taxane chemotherapeutics (Antonarakis, E. & Paller, E. S. 2011; Cella, D. et al. 2003; Baker, J. et al. 2009; Sperlich, C. & Saad, F. 2013).

Fatty acid-binding protein 5 (FABP5) is an intracellular lipid carrier whose expression is upregulated in metastatic PCa and increases cell growth, invasion, and tumor formation. FABP5 inhibitors based upon the truxillic acid monoester scaffold have been developed, including the first-generation inhibitor Stony Brook fatty acid-binding protein inhibitor 26 (SBFI-26) (Berger, W. T. et al. 2012; Kaczocha, M. et al. 2014). FABP5 inhibitors that show enhanced potency or selectivity for FABP5 have been identified (Yan, S. et al. 2018). SBFI-26 suppresses PCa cell growth, migration, invasion, tumor formation, and metastasis in vitro and in vivo (Al-Jameel, W. et al. 2017), suggesting that FABP5 inhibitors may constitute efficacious antitumor agents. Thus, we assessed whether FABP5 inhibitors synergize with clinically used taxanes to induce cytotoxicity in vitro and attenuate tumor growth in vivo.

We found that SBFI-102 and SBFI-103 produced cytotoxicity in the PCa cells. Coincubation of the PCa cells with FABP5 inhibitors and docetaxel or cabazitaxel produced synergistic cytotoxic effects in vitro. Treatment of mice with FABP5 inhibitors reduced tumor growth and a combination of FABP5 inhibitors with a submaximal dose of docetaxel reduced tumor growth to a larger extent than treatment with each drug alone. Thus, FABP5 inhibitors increase the cytotoxic and tumor-suppressive effects of taxanes in PCa cells. The ability of these drugs to synergize could permit more efficacious antitumor activity while allowing for dosages of docetaxel or cabazitaxel to be lowered, potentially decreasing taxane-resistance as well as taxane-associated toxicity.

### REFERENCES

Adamson, J., Morgan, E. A., Beesley, C., et al. (2003) High-level expression of cutaneous fatty acid-binding protein in prostatic carcinomas and its effect on tumorigenicity. Oncogene 22, 2739-2749.
Ahmad, I., Mui, E., Galbraith, L., Patel, R., Tan, E. H., Salji, M., Rust, A. G., Repiscak, P., Hedley, A., Markert, E., Loveridge, C., van der Weyden, L., Edwards, J., Sansom, O. J., Adams, D. J., Leung, H. Y. (2016) Sleeping Beauty screen reveals Pparg activation in metastatic prostate cancer. Proc Natl Acad Sci USA, 113, 8290-8295.
Al-Jameel, W., Gou, X., Forootan, S. S., et al. (2017) Inhibitor SBFI26 suppresses the malignant progression of castration-resistant PC3-M cells by competitively binding to oncogenic FABP5. Oncotarget 8, 31041-31056.
Antonarakis, E., Paller, E. S. (2011) Cabazitaxel: a novel second-line treatment for metastatic castration-resistant prostate cancer. Drug Des Devel Ther. 5, 117-124.
Baker, J., Ajani, J., Scotte, F., et al. (2009) Docetaxel-related side effects and their management. Eur J Oncol Nurs. 13, 49-59.
Bao, Z., Malki, M. I., Forootan, S. S., Adamson, J., Forootan, F. S., Chen, D., Foster, C. S., Rudland, P. S., Ke, Y. (2013) A novel cutaneous Fatty Acid-binding protein-related signaling pathway leading to malignant progression in prostate cancer cells. Genes Cancer 4, 297-314.
Berger, W. T., Ralph, B. P., Kaczocha, M., Sun, J., Balius, T. E., Rizzo, R. C., Haj-Dahmane, S., Ojima, I., and Deutsch, D. G. (2012) Targeting fatty acid binding protein (FABP) anandamide transporters—a novel strategy for development of anti-inflammatory and anti-nociceptive drugs. PLoS One 7, e50968.
de Bono, J. S., Oudard, S., Ozguroglu, M., et al. (2010) Prednisone plus cabazitaxel or mitoxantrone for metastatic castration-resistant prostate cancer progressing after docetaxel treatment: a randomized open-label trial. The Lancet 376, 1147-1154.
Bray, F., Ferlay, J., Soerjomataram, I., Siegel, R. L., Torre, L. A., Jemal, A. (2018) Global cancer statistics 2018: GLOBOCAN estimates of incidence and mortality worldwide for 36 cancers in 185 countries. CA Cancer J Clin 68, 394-424.
Brod et al. (2000) Annals of Neurology, 47:127-131.
Cella, D., Peterman, A., Hudgens, S., Webster, K., Socinski, M. A. (2003) Measuring the side effects of taxane therapy in oncology: The Functional Assessment of Cancer Therapy-taxane (FACT-taxane). Cancer 98, 822-831.
Chou, T. C. (2006) Theoretical basis, experimental design, and computerized simulation of synergism and antagonism in drug combination studies. Pharmacol Rev. 58, 621-681.
Deep, G., Schlaepfer, I. (2016) Aberrant lipid metabolism promotes prostate cancer: role in cell survival under hypoxia and extracellular vesicles biogenesis. Int J Mol Sci. 17, 1061.
Forootan, F. S., Forootan, S. S., Gou, X., et al. (2016) Fatty acid activated PPARgamma promotes tumorigenicity of prostate cancer cells by up regulating VEGF via PPAR responsive elements of the promoter. Oncotarget 7, 9322-9339.
Forootan, F. S., Forootan, S. S., Malki, M. I., Chen, D., Li, G., Lin, K., Rudland, P. S., Foster, C. S., Ke, Y. (2014) The expression of C-FABP and PPARγ and their prognostic significance in prostate cancer. Int J Oncol 44, 265-275.
Frieling, J. S., Basanta, D., Lynch, C. C. (2015) Current and emerging therapies for bone metastatic castration-resistant prostate cancer. Cancer Control 22, 109-120.
Fujita, K., Kume, H., Matsuzaki, K., Kawashima, A., Ujike, T., Nagahara, A., Uemura, M., Miyagawa, Y., Tomonaga, T., Nonomura, N. (2017) Proteomic analysis of urinary extracellular vesicles from high Gleason score prostate cancer. Sci rep. 7, 42961.
Furuhashi, M. and Hotamisligil, G. S. (2008) Fatty acid-binding proteins: role in metabolic diseases and potential as drug targets. Nat Rev Drug Discov 7, 489-503.
Galletti, G., Leach, B. I., Lam, L., Tagawa, S. T. (2017) Mechanisms of resistance to systemic therapy in metastatic castration-resistant prostate cancer. Cancer Treat Rev. 57, 16-27.
Guidance for Industry. In vivo drug metabolism/drug interaction studies—study design, data analysis, and recommendations for dosing and labeling, U.S. Dept. Health and Human Svcs., FDA, Ctr. for Drug Eval. and Res., Ctr. For Biologics Eval. and Res., Clin. Pharm., November 1999 <http://www.fda.gov/cber/gdlns/metabol.pdf>.
Higano, C. S., Crawford, E. D. (2011) New and emerging agents for the treatment of castration-resistant prostate cancer. Urol Oncol. 29, 1-8.
Hongo, H., Kosaka, T., Oya, M. (2018) Analysis of cabazitaxel-resistant mechanism in human castration-resistant prostate cancer. Cancer Sci. 109, 2937-2945.
Jing, C., Beesley, C., Foster, C. S., et al. (2000) Identification of the messenger RNA for human cutaneous fatty acid-binding protein as a metastasis inducer. Cancer Res. 60, 2390-2398.
Kaczocha, M., Rebecchi, M. J., Ralph, B. P., et al. (2014) Inhibition of fatty acid binding proteins elevates brain anandamide levels and produces analgesia. PLoS One 9, e94200.
Kawaguchi, K., Kinameri, A., Suzuki, S., Senga, S., Ke, Y., Fujii, H. (2016) The cancer-promoting gene fatty acid-binding protein 5 (FABP5) is epigenetically regulated during human prostate carcinogenesis. Biochem J. 473, 449-461.
Kleinschmidt-DeMasters et al. (2005) New England Journal of Medicine, 353:369-379.
Langer-Gould et al. (2005) New England Journal of Medicine, 353:369-379.
Morgan, E. A., Forootan, S. S., Adamson, J., et al. (2008) Expression of cutaneous fatty acid-binding protein (C-FABP) in prostate cancer: potential prognostic marker and target for tumourigenicity-suppression. Int J Oncol. 32, 767-775.
Rudick et al. (2006) New England Journal of Medicine, 354:911-923.
Sperlich, C., Saad, F. (2013) Optimal management of patients receiving cabazitaxel-based chemotherapy. Can Urol Assoc J. 7, S18-S24.
Tannock, I. F., de Wit, R., Berry, W. R., et al. (2004) Docetaxel plus prednisone or mitoxantrone plus prednisone for advanced prostate cancer. N Engl J Med. 351, 1502-1512.
Vollmer et al. (2008) “Glatiramer acetate after induction therapy with mitoxantrone in relapsing multiple sclerosis” Multiple Sclerosis, 00:1-8.
Yan, S., Elmes, M. W., Tong, S., et al. (2018) SAR studies on truxillic acid mono esters as a new class of antinociceptive agents targeting fatty acid binding proteins. Eur J Med Chem. 154, 233-252.
Zadra, G., Photopoulos, C., Loda, M. (2013) The fat side of prostate cancer. Biochim Biophys Acta. 1831, 1518-1532.

1. A method of treating cancer in a subject comprising administering to the subject an effective amount of a FABP5 inhibitor with an anticancer therapy.

2. The method of claim 1 comprising periodically administering to the subject an amount of the FABP5 inhibitor and the anticancer therapy, wherein the amounts when taken together are effective to treat the subject, or
wherein the amount of the FABP5 inhibitor, and the amount of the anticancer therapy when administered together is more effective to treat the subject than when each agent at the same amount is administered alone.

3. (canceled)

4. The method of claim 1, wherein the subject is receiving the anticancer therapy prior to initiating the FABP5 inhibitor therapy, or
wherein the subject is receiving the FABP5 inhibitor therapy prior to initiating the anticancer therapy, or
wherein the FABP5 inhibitor, and the anticancer therapy are administered sequentially, or
wherein the FABP5 inhibitor, and the anticancer therapy are administered simultaneously.

5. (canceled)

6. (canceled)

7. The method of claim 1 wherein the FABP5 inhibitor is administered first, followed by administration of the anticancer therapy, or
wherein the anticancer therapy is administered first, followed by administration of the FABP5 inhibitor.

8. (canceled)

9. (canceled)

10. The method of claim 1, wherein the FABP5 inhibitor is administered orally, intravenously, or intraperitoneally.

11. The method of claim 1, wherein the anticancer therapy is a taxane,
wherein the taxane is administered intravenously or intraperitoneally.

12. (canceled)

13. The method of claim 1, wherein the cancer expresses FABP5, or
wherein the cancer overexpresses FABP5.

14. (canceled)

15. The method of claim 1, wherein the cancer is prostate cancer, drug-resistant prostate cancer, metastatic prostate cancer, skin cancer, breast cancer, hepatocellular carcinoma or cervical cancer.

16. (canceled)

17. (canceled)

18. (canceled)

19. The method of claim 1, wherein the FABP5 inhibitor has the structure: 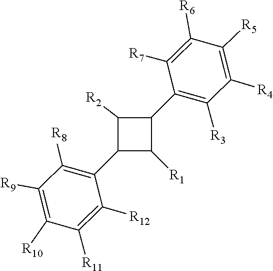
wherein
R₁ and R₂ are different and are each —C(═O)R₁₃, —C(═O)OR₁₃, —C(═O)O-alkyl-R₁₃, —C(═O)NR₁₃R₁₄, -alkyl-C(═O)R₁₃, -alkyl-C(═O)OR₁₃, -alkyl-C(═O)NR₁₃R₁₄, -alkyl-OC(═O)OR₁₃, -alkyl-OC(═O)R₁₃, -alkyl-OR₁₃, -alkyl-NR₁₃R₁₄, -alkyl-NHC(═O)R₁₃, -alkyl-NHC(═O)OR₁₃, -alkyl-NHC(═O)R₁₃, -alkyl-NHC(═O)NR₁₃R₁₄, -alkyl-NHC(═S)NR₁₃R₁₄, -alkyl-NHC(═NR₁₃)NR₁₃R₁₄, —C(—OH)C(═O)OR₁₃, —C(═O) C(═O)OR₁₃ or —C═C—R₁₃,
wherein R₁₃ and R₁₄ are each, independently, H, CF₃, C₁₋₁₀ alkyl, C₂₋₁₀ alkenyl, C₂₋₁₀ alkynyl, heteroalkyl, cycloalkyl, cycloheteroalkyl, aryl, heteroaryl, heterocyclyl or combine to form a cycloalkyl or heterocyclyl;
R₃, R₄, R₅, R₆, R₇, R₈, R₉, R₁₀, R₁₁ and R₁₂ are each independently, H, halogen, —NO₂, —CN, —NHR₁₅, —NR₁₅R₁₆, —SR₁₅, —SO₂R₁₅, —OR_{Th}, —CO₂R₁₅, CF₃, -alkyl-NR₁₅R₁₆, -alkyl-OR₁₅, C₂₋₂₀ alkyl, C₂₋₁₀ alkenyl, C₂₋₁₀ alkynyl, aryl, heteroaryl, or heterocyclyl;
wherein R₁₅ and R₁₆ are each, independently, H, CF₃, C₁₋₁₀ alkyl, C₂₋₁₀ alkenyl, C₂₋₁₀ alkynyl, heteroalkyl, cycloheteroalkyl, aryl, heteroaryl, or heterocyclyl;
wherein when the compound has the stereochemistry of structure I 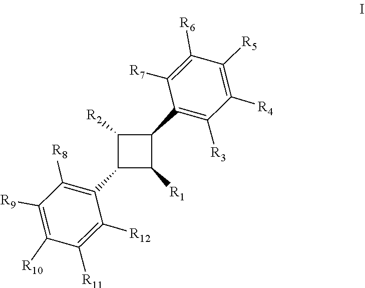
then
R₁ and R₂ are different and are each —C(═O)R₁₃, —C(═O)OR₁₃, —C(═O)O-alkyl-R₁₃, —C(═O)NR₁₃R₁₄, -alkyl-C(═O)R₁₃, -alkyl-C(═O)OR₁₃, -alkyl-C(═O)NR₁₃R₁₄, -alkyl-OC(═O)OR₁₃, -alkyl-OC(═O)R₁₃, -alkyl-OR₁₃, -alkyl-NR₁₃R₁₄, -alkyl-NHC(═O)R₁₃, -alkyl-NHC(═O)OR₁₃, -alkyl-NHC(═O)R₁₃, -alkyl-NHC(═O)NR₁₃R₁₄, -alkyl-NHC(═S)NR₁₃R₁₄, -alkyl-NHC(═NR₁₃)NR₁₃R₁₄, —C(—OH)C(═O)OR₁₃, —C(═O) C(═O)OR₁₃ or —C═C—R₁₃,
wherein R₁₃ and R₁₄ are each, independently, H, CF₃, C₁₋₁₀ alkyl, C₂₋₁₀ alkenyl, C₂₋₁₀ alkynyl, heteroalkyl, cycloalkyl, cycloheteroalkyl, aryl, heteroaryl, heterocyclyl or combine to form a cycloalkyl or heterocyclyl;
R₃, R₄, R₅, R₆, R₇, R₈, R₉, R₁₀, R₁₁ and R₁₂ are each independently, H, halogen, —NO₂, —CN, —NHR₁₅, —NR₁₅R₁₆, —SR₁₅, —SO₂R₁₅, —OR_{Th}, —CO₂R₁₅, CF₃, -alkyl-NR₁₅R₁₆, -alkyl-OR₁₅, C₂₋₂₀ alkyl, C₂₋₁₀ alkenyl, C₂₋₁₀ alkynyl, aryl, heteroaryl, or heterocyclyl;
wherein R₁₅ and R₁₆ are each, independently, H, CF₃, C₁₋₁₀ alkyl, C₂₋₁₀ alkenyl, C₂₋₁₀ alkynyl, heteroalkyl, cycloheteroalkyl, aryl, heteroaryl, or heterocyclyl;
wherein when the compound has the stereochemistry of structure II 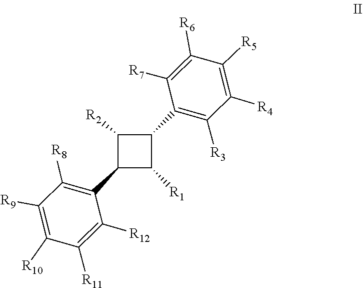
then
R₁ and R₂ are different and are each —C(═O)R₁₃, —C(═O)OR₁₃, —C(═O)O-alkyl-R₁₃, —C(═O)NR₁₃R₁₄, -alkyl-C(═O)R₁₃, -alkyl-C(═O)OR₁₃, -alkyl-C(═O)NR₁₃R₁₄, -alkyl-OC(═O)OR₁₃, -alkyl-OC(═O)R₁₃, -alkyl-OR₁₃, -alkyl-NR₁₃R₁₄, -alkyl-NHC(═O)R₁₃, -alkyl-NHC(═O)OR₁₃, -alkyl-NHC(═O)R₁₃, -alkyl-NHC(═O)NR₁₃R₁₄, -alkyl-NHC(═S)NR₁₃R₁₄, -alkyl-NHC(═NR₁₃)NR₁₃R₁₄, —C(—OH)C(═O)OR₁₃, —C(═O) C(═O)OR₁₃ or —C═C—R₁₃,
wherein R₁₃ and R₁₄ are each, independently, H, CF₃, C₁₋₁₀ alkyl, C₂₋₁₀ alkenyl, C₂₋₁₀ alkynyl, heteroalkyl, cycloalkyl, cycloheteroalkyl, aryl, heteroaryl, heterocyclyl or combine to form a cycloalkyl or heterocyclyl;
R₃, R₄, R₅, R₆, R₇, R₈, R₉, R₁₀, R₁₁ and R₁₂ are each independently, H, halogen, —NO₂, —CN, —NHR₁₅, —NR₁₅R₁₆, —SR₁₅, —SO₂R₁₅, —OR₁₅, —CO₂R₁₅, CF₃, -alkyl-NR₁₅R₁₆, -alkyl-OR₁₅, C₁₋₁₀ alkyl, C₂₋₁₀ alkenyl, C₂₋₁₀ alkynyl, aryl, heteroaryl, or heterocyclyl;
wherein R₁₅ and R₁₆ are each, independently, H, CF₃, C₁₋₁₀ alkyl, C₂₋₄₀ alkenyl, C₂₋₁₀ alkynyl, heteroalkyl, cycloheteroalkyl, aryl, heteroaryl, or heterocyclyl;
or an enantiomer or racemate thereof;
or a pharmaceutically acceptable salt thereof.

20. (canceled)

21. The method of claim 1, wherein the FABP5 inhibitor has the structure: 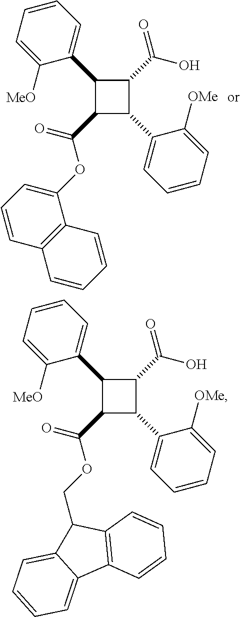
or a pharmaceutically acceptable salt thereof.

22. The method of claim 1, wherein the taxane is paclitaxel, docetaxel, or cabazitaxel.

23. (canceled)

24. (canceled)

25. (canceled)

26. The method of claim 1, wherein the anticancer therapy is radiation therapy,
wherein the radiation therapy is external beam radiation or brachytherapy.

27. (canceled)

28. (canceled)

29. The method of claim 1, wherein the subject is a mammal.

30. A pharmaceutical composition comprising a FABP5 inhibitor of claim 9 and a pharmaceutically acceptable carrier.

31. (canceled)

32. (canceled)

33. The pharmaceutical composition of claim 14 wherein the FABP5 inhibitor has the structure:

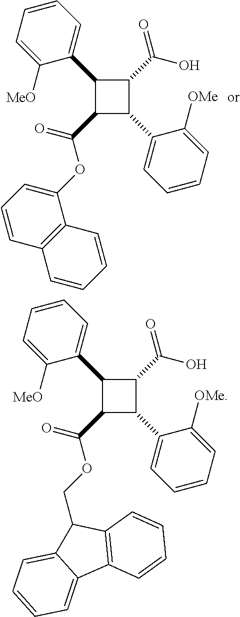

34. The pharmaceutical composition of claim 14, further comprising a taxane,
wherein the taxane is docetaxel or cabazitaxel.

35. (canceled)

36. The use of a FABP5 inhibitor of claim 9 in combination or as an add on with an anticancer therapy in treating a subject afflicted with cancer, wherein the FABP5 inhibitor and the anticancer therapy are administered simultaneously, contemporaneously or concomitantly.

37. (canceled)

38. The use of claim 17, wherein the anticancer therapy is a taxane or radiation therapy,
wherein the cancer is prostate cancer.

39. (canceled)

40. (canceled)

41. (canceled)

42. A pharmaceutical composition comprising an amount of a FABP5 inhibitor of claim 9 and an amount of an anticancer therapy for use in treating a subject afflicted with cancer, wherein the FABP5 inhibitor and the anticancer therapy are administered simultaneously, contemporaneously or concomitantly.

43. (canceled)

44. The pharmaceutical composition of claim 19, wherein the anticancer therapy is a taxane or radiation therapy,
wherein the cancer is prostate cancer.

45. (canceled)

46. (canceled)
